(19) United States
(12) Patent Application Publication
(10) Pub. No.: US 2026/0108581 A1
(43) Pub. Date: Apr. 23, 2026

(54) COMPOSITIONS AND METHODS FOR MICROSPHERE DELIVERY OF CARNOSINE FOR TREATMENT OF NEURODEGENERATIVE DISEASES AND DISORDERS

(71) Applicant: Finitus, LLC, St. Petersburg, FL (US)

(72) Inventors: Spencer Bouhadir, Lake Worth, FL (US); Jacob J. Miguel, Pinecrest, FL (US)

(21) Appl. No.: 19/070,270

(22) Filed: Mar. 4, 2025

Related U.S. Application Data
(60) Provisional application No. 63/710,787, filed on Oct. 23, 2024.
(60) Provisional application No. 63/728,055, filed on Dec. 4, 2024.
(60) Provisional application No. 63/728,050, filed on Dec. 4, 2024.
(60) Provisional application No. 63/759,959, filed on Feb. 18, 2025.
(60) Provisional application No. 63/759,955, filed on Feb. 18, 2025.
(60) Provisional application No. 63/759,942, filed on Feb. 18, 2025.

(51) Int. Cl. A61K 38/05; A61K 9/00; A61K 9/50; A61P 25/16

(52) U.S. Cl. CPC A61K 38/05; A61K 9/0019; A61K 9/5026; A61K 9/5031; A61K 9/5036; A61P 25/16

(57) ABSTRACT

The present disclosure provides, inter alia, compositions and methods for microsphere delivery of carnosine for treatment of neurodegenerative diseases or conditions.

### CROSS-REFERENCE TO RELATED APPLICATIONS

The present application claims priority to and benefit of U.S. provisional application Ser. No. 63/710,787 filed Oct. 23, 2024, 63/728,055 filed Dec. 4, 2024, 63/728,050 filed Dec. 4, 2024, 63/759,959 filed Feb. 18, 2025, 63/759,955 filed Feb. 18, 2025, 63/759,942 filed Feb. 18, 2025, the entire contents of which are incorporated by reference.

### FIELD OF INVENTION

The present disclosure relates generally to microsphere formulations of carnosine for treatment of neurological conditions.

### BACKGROUND OF THE INVENTION

Neurological and neurodegenerative diseases and disorders represent potentially debilitating conditions that can affect people of all ages and may be acquired, congenital, hereditary, or sporadic conditions. They are typically associated with widely varying degrees of mental, emotional, physical, and economic consequences for individuals.

Neurodegenerative diseases and disorders are typically characterized by progressive nervous system dysfunction and may be associated with atrophy of the affected central or peripheral structures of the nervous system. Examples of neurodegenerative diseases and disorders include but are not limited to dementia, including Alzheimer's Disease, degenerative nerve diseases, encephalitis, epilepsy, hydrocephalus, stroke, Parkinson's disease, multiple sclerosis, brain cancer, cognitive dysfunction syndrome, amyotrophic lateral sclerosis (ALS), and Huntington's disease.

One common neurodegenerative disease is Alzheimer's disease. Alzheimer's disease is a highly variable condition which presents and develops differently in each individual. While the cause and progression of the disease is not well understood, there are many common symptoms. In the initial stages, sufferers often exhibit short term memory loss. As the disease progresses, symptoms may include confusion, irritability, aggression, mood swings, trouble with language, and long-term memory loss. The later stages are often characterized by loss of bodily functions which ultimately results to death.

Another common neurodegenerative disease is Parkinson's disease. Parkinson's disease is a progressive movement disorder of the nervous system, wherein nerve cells (neurons) in parts of the brain to weaken, become damaged, and die. This leads to symptoms that include problems with movement, tremor, stiffness, and impaired balance. As symptoms progress, people with Parkinson's disease (PD) may have difficulty walking, talking, or completing other simple tasks.

One therapeutic agent that shows promise for treatment of neurological diseases or conditions (including Alzheimer's and Parkinson's) is carnosine. Carnosine is a dipeptide of β-alanine and histidine that naturally occurs in many mammalian tissues including brain and muscle. Carnosine has been identified as providing neuroprotective functions of brain tissue via many mechanisms that are not fully understood. For example, carnosine had been shown to facilitate lactate export from cells and, hence, provide metabolic support for neurons and axons by buffering protons. Furthermore, well-known peripheral actions of carnosine have also been reported in the brain, such as reductions in oxidative stress, inflammation, and advanced glycation end products as well as regulation of macrophage function. Other observed neuroprotective actions of carnosine include attenuation of neurotoxicity induced by NMDA, beta amyloid, and inducible nitric oxide synthase as well as increased trophic factors expression.

While carnosine is capable of passing through the blood brain barrier (BBB), it is currently understood that the majority of brain carnosine is a product of its de novo synthesis localized to specific areas of the brain rather than a result of its penetration through the BBB. Moreover, carnosine is metabolized rapidly in the body due to the presence of both serum and tissue carnosinase enzymes. Thus, its use as a supplement for treatment of neurological diseases or disorders would require administration of multiple daily doses, which is impractical.

Thus, there exists a need to develop formulations for the treatment of neurodegenerative diseases and disorders that provides sustained delivery of carnosine with a single administration. The present disclosure is directed to meeting these and other needs.

### SUMMARY OF THE INVENTION

According to some aspects, the present disclosure provides a microsphere formulation effective for treatment of a neurological disease or condition comprising a plurality of microspheres comprising carnosine; a biodegradable polymer; and cyclodextrin; wherein the carnosine is entrapped by the biodegradable polymer and cyclodextrin; and wherein the microspheres have an average size of less than 100 μm.

In some embodiments, the carnosine is one or more of L-carnosine, D-carnosine, acetyl-carnosine, and combinations thereof. In some embodiments, the biodegradable polymer comprises one or more of poly(D,L-lactide), a poly(D,L-lactide-co-glycolide), a poly(ortho ester), a poly(phosphazine), a poly(phosphate ester), a polycaprolactone, a polyethylene glycol, sodium-gamma-polyglutamate, hyaluornic acid, and combinations thereof. In some embodiments, the biodegradable polymer is a poly(D,L-lactide-co-glycolide). In some embodiments, the biodegradable polymer is a poly(D,L-lactide-co-glycolide) having a lactide to glycolide ratio of about 75:25. In some embodiments, the biodegradable polymer is a poly(D,L-lactide-co-glycolide) having a lactide to glycolide ratio of about 50:50. In some embodiments, the biodegradable polymer is a poly(D,L-lactide-co-glycolide) having a lactide to glycolide ratio of between about 75:25 to about 50:50.

In some embodiments, the microsphere further comprises one or more of polyvinyl alcohol, polyanhydrides, polyamines, polyesteramides, polydioxanones, polyacetals, polyketals, polycarbonates, polyphosphesters, polyethers, polyesters, polybutylene, terephthalate, polyorthocarbonates, polyphosphazenes, succinates, poly(malic acid), poly(amino acids), polyvinypyrrolidone, polysaccharides, oligosaccharides, alginate copolymers, terpolymers and combinations thereof.

In some embodiments, the microspheres have an average size of 10 μm to 50 μm. In some embodiments, the microspheres have an average size of 10 μm to 50 μm wherein 90% of the microspheres have a size between 1 μm and 100 μm.

In some embodiments, the cyclodextrin is an alpha cyclodextrin, a beta cyclodextrin, a gamma cyclodextrin, a 2-hydroxypropyl-β-cyclodextrin, a β-cyclodextrin sulfobutylether, hydroxyethyl-β-cyclodextrin, methyl-β-cyclodextrin, dimethyl-β-cyclodextrin, carboxymethyl-β-cyclodextrin, carboxymethyl ethyl-β-cyclodextrin, diethyl-β-cyclodextrin, tri-O-alkyl-1-β-cyclodextrin, glycosyl-β-cyclodextrin, maltosyl-β-cyclodextrin or any derivative thereof, and any combination thereof. In some embodiments, the cyclodextrin is a gamma cyclodextrin. In some embodiments, the microsphere comprises 1%-20% cyclodextrin by weight. In some embodiments, the microsphere comprises 1%-80% carnosine by weight. In some embodiments, the microsphere comprises 1%-80% biodegradable polymer by weight.

According to some aspects, the present disclosure provides a microsphere effective for treatment of a neurological disease or condition produced by the process of providing an aqueous phase comprising carnosine in an aqueous solution; providing an organic phase comprising a biodegradable polymer and cyclodextrin in an organic solution; combining the organic phase with the aqueous phase to produce a primary emulsion; providing an external aqueous phase; combining the primary emulsion with the external aqueous phase to produce a double emulsion; and evaporating the solvents to produce the microsphere formulation.

In some embodiments, the aqueous phase further comprises one or more of polyvinyl alcohol, polyanhydrides, polyamines, polyesteramides, polydioxanones, polyacetals, polyketals, polycarbonates, polyphosphesters, polyethers, polyesters, polybutylene, terephthalate, polyorthocarbonates, polyphosphazenes, succinates, poly(malic acid), poly(amino acids), polyvinypyrrolidone, polysaccharides, oligosaccharides, alginate copolymers, terpolymers and combinations thereof.

In some embodiments, the organic phase biodegradable polymer comprises one or more of poly(D,L-lactide), a poly(D,L-lactide-co-glycolide), a poly(ortho ester), a poly(phosphazine), a poly(phosphate ester), a polycaprolactone, a polyethylene glycol, sodium-gamma-polyglutamate, hyaluornic acid, and combinations thereof. In some embodiments, the organic phase biodegradable polymer comprises a poly(D,L-lactide-co-glycolide). In some embodiments, the organic phase biodegradable polymer comprises a poly(D,L-lactide-co-glycolide) having a lactide to glycolide ratio of about 75:25. In some embodiments, the organic phase biodegradable polymer comprises a poly(D,L-lactide-co-glycolide) having a lactide to glycolide ratio of about 50:50. In some embodiments, the cyclodextrin is an alpha cyclodextrin, a beta cyclodextrin, a gamma cyclodextrin, a 2-hydroxypropyl-β-cyclodextrin, a β-cyclodextrin sulfobutylether, hydroxyethyl-β-cyclodextrin, methyl-β-cyclodextrin, dimethyl-β-cyclodextrin, carboxymethyl-β-cyclodextrin, carboxymethyl ethyl-β-cyclodextrin, diethyl-β-cyclodextrin, tri-O-alkyl-1-β-cyclodextrin, glycosyl-β-cyclodextrin, maltosyl-β-cyclodextrin or any derivative thereof, and any combination thereof. In some embodiments, the cyclodextrin is a gamma cyclodextrin.

In some embodiments, the microsphere comprises 1%-20% cyclodextrin by weight. In some embodiments, the microsphere comprises 1%-80% carnosine by weight. In some embodiments, the microsphere comprises 1%-80% biodegradable polymer by weight.

According to some aspects, the present disclosure provides a method of treating or ameliorating a neurodegenerative disease or condition in a subject in need thereof comprising the step administering to the subject the formulation disclosed herein or the microsphere disclosed herein. In some embodiments, the formulation or microsphere is administered orally, intramuscularly, intravenously, subcutaneously, intrathecally, and/or by nasal spray. In some embodiments, the formulation or microsphere is administered no more than once per week. In some embodiments, the formulation or microsphere is administered no more than once per month. In some embodiments, the formulation or microsphere is administered no more than once every six months.

According to some aspects, the present disclosure provides a method of making a microsphere effective for treatment of a neurological disease or comprising the steps of providing an aqueous phase comprising carnosine in an aqueous solution; providing an organic phase comprising a biodegradable polymer and cyclodextrin in an organic solution; combining the organic phase with the aqueous phase to produce a primary emulsion; providing an external aqueous phase; combining the primary emulsion with the external aqueous phase to produce a double emulsion; and evaporating the solvents to produce the microsphere formulation.

In some embodiments, the aqueous phase further comprises one or more of polyvinyl alcohol, polyanhydrides, polyamines, polyesteramides, polydioxanones, polyacetals, polyketals, polycarbonates, polyphosphesters, polyethers, polyesters, polybutylene, terephthalate, polyorthocarbonates, polyphosphazenes, succinates, poly(malic acid), poly(amino acids), polyvinypyrrolidone, polysaccharides, oligosaccharides, alginate copolymers, terpolymers and combinations thereof. In some embodiments, the organic phase biodegradable polymer comprises one or more of poly(D,L-lactide), a poly(D,L-lactide-co-glycolide), a poly(ortho ester), a poly(phosphazine), a poly(phosphate ester), a polycaprolactone, a polyethylene glycol, sodium-gamma-polyglutamate, hyaluornic acid, and combinations thereof. In some embodiments, the organic phase biodegradable polymer comprises a poly(D,L-lactide-co-glycolide). In some embodiments, the organic phase biodegradable polymer comprises a poly(D,L-lactide-co-glycolide) having a lactide to glycolide ratio of about 75:25. In some embodiments, the organic phase biodegradable polymer comprises a poly(D,L-lactide-co-glycolide) having a lactide to glycolide ratio of about 50:50.

In some embodiments, the cyclodextrin is an alpha cyclodextrin, a beta cyclodextrin, a gamma cyclodextrin, a 2-hydroxypropyl-β-cyclodextrin, a β-cyclodextrin sulfobutylether, hydroxyethyl-β-cyclodextrin, methyl-β-cyclodextrin, dimethyl-β-cyclodextrin, carboxymethyl-β-cyclodextrin, carboxymethyl ethyl-β-cyclodextrin, diethyl-β-cyclodextrin, tri-O-alkyl-1-β-cyclodextrin, glycosyl-β-cyclodextrin, maltosyl-β-cyclodextrin or any derivative thereof, and any combination thereof. In some embodiments, the cyclodextrin is a gamma cyclodextrin. In some embodiments, the microsphere comprises 1%-20% cyclodextrin by weight. In some embodiments, the microsphere comprises 1%-80% carnosine by weight. In some embodiments, the microsphere comprises 1%-80% biodegradable polymer by weight. The formulation of claim 1, wherein the microspheres are effective to encapsulate at least 20% of the peptide.

In some embodiments, the microspheres disclosed herein are effective to encapsulate at least 20% of the peptide. In some embodiments, the microspheres disclosed herein are effective to encapsulate at least 40% of the peptide. In some embodiments, the microspheres disclosed herein are effective to encapsulate at least 60% of the peptide. In some embodiments, the microspheres disclosed herein are effective to encapsulate at least 80% of the peptide. In some embodiments, the methods disclosed herein provide microspheres that are effective to encapsulate at least 20% of the peptide. In some embodiments, the methods disclosed herein provide microspheres that are effective to encapsulate at least 40% of the peptide. In some embodiments, the methods disclosed herein provide microspheres that are effective to encapsulate at least 60% of the peptide. In some embodiments, the methods disclosed herein provide microspheres that are effective to encapsulate at least 80% of the peptide.

In some embodiments, the microspheres disclosed herein further comprise a lyoprotectant. In some embodiments, the methods disclosed herein provide a microsphere further comprising a lyoprotectant.

According to some aspects, the present disclosure provides a method of treating or ameliorating a neurodegenerative disease or condition in a subject in need thereof comprising the step of administering to the subject a microsphere formulation comprising: a plurality of microspheres comprising carnosine; a biodegradable polymer; and cyclodextrin; wherein the carnosine is entrapped by the biodegradable polymer and cyclodextrin; and wherein the microspheres have an average size of less than 100 μm.

In some embodiments, the carnosine is one or more of L-carnosine, D-carnosine, acetyl-carnosine, and combinations thereof. In some embodiments, the biodegradable polymer comprises one or more of poly(D,L-lactide), a poly(D,L-lactide-co-glycolide), a poly(ortho ester), a poly(phosphazine), a poly(phosphate ester), a polycaprolactone, a polyethylene glycol, sodium-gamma-polyglutamate, hyaluornic acid, and combinations thereof. In some embodiments, the biodegradable polymer is a poly(D,L-lactide-co-glycolide). In some embodiments, the biodegradable polymer is a poly(D,L-lactide-co-glycolide) having a lactide to glycolide ratio of about 75:25. In some embodiments, the biodegradable polymer is a poly(D,L-lactide-co-glycolide) having a lactide to glycolide ratio of about 50:50.

In some embodiments, the microsphere further comprises one or more of polyvinyl alcohol, polyanhydrides, polyamines, polyesteramides, polydioxanones, polyacetals, polyketals, polycarbonates, polyphosphesters, polyethers, polyesters, polybutylene, terephthalate, polyorthocarbonates, polyphosphazenes, succinates, poly(malic acid), poly(amino acids), polyvinypyrrolidone, polysaccharides, oligosaccharides, alginate copolymers, terpolymers and combinations thereof.

In some embodiments, the microspheres have an average size of 10 μm to 50 μm. In some embodiments, the microspheres have an average size of 10 μm to 50 μm wherein 90% of the microspheres have a size between 1 μm and 100 μm. In some embodiments, the cyclodextrin is an alpha cyclodextrin, a beta cyclodextrin, a gamma cyclodextrin, a 2-hydroxypropyl-β-cyclodextrin, a β-cyclodextrin sulfobutylether, hydroxyethyl-β-cyclodextrin, methyl-β-cyclodextrin, dimethyl-β-cyclodextrin, carboxymethyl-β-cyclodextrin, carboxymethyl ethyl-β-cyclodextrin, diethyl-β-cyclodextrin, tri-O-alkyl-1-β-cyclodextrin, glycosyl-β-cyclodextrin, maltosyl-β-cyclodextrin or any derivative thereof, and any combination thereof.

In some embodiments, the cyclodextrin is a gamma cyclodextrin. In some embodiments, the microsphere comprises 1%-20% cyclodextrin by weight. In some embodiments, the microsphere comprises 1%-80% carnosine by weight. In some embodiments, the microsphere comprises 1%-80% biodegradable polymer by weight.

In some embodiments, the formulation is administered orally, intramuscularly, intravenously, subcutaneously, intrathecally, and/or by nasal spray. In some embodiments, the formulation is administered no more than once per week. In some embodiments, the formulation is administered no more than once per month. In some embodiments, the formulation is administered no more than once every six months.

In some embodiments, the neurodegenerative disease or condition is Alzheimer's disease. In some embodiments, the neurodegenerative disease or condition is Parkinson's disease.

In some embodiments, the microspheres are effective to encapsulate at least 20% of the carnosine. In some embodiments, the microspheres are effective to encapsulate at least 40% of the carnosine. In some embodiments, the microspheres are effective to encapsulate at least 60% of the carnosine. In some embodiments, the microspheres are effective to encapsulate at least 80% of the carnosine.

According to some aspects, the present disclosure provides a microsphere effective for treatment of a neurodegenerative disease or condition produced by the steps of: providing an aqueous phase comprising carnosine in an aqueous solution; providing an organic phase comprising a biodegradable polymer and cyclodextrin in an organic solution; combining the organic phase with the aqueous phase to produce a primary emulsion; providing an external aqueous phase; combining the primary emulsion with the external aqueous phase to produce a double emulsion; and evaporating the solvents to produce the microsphere formulation.

In some embodiments, the aqueous phase further comprises one or more of polyvinyl alcohol, polyanhydrides, polyamines, polyesteramides, polydioxanones, polyacetals, polyketals, polycarbonates, polyphosphesters, polyethers, polyesters, polybutylene, terephthalate, polyorthocarbonates, polyphosphazenes, succinates, poly(malic acid), poly(amino acids), polyvinypyrrolidone, polysaccharides, oligosaccharides, alginate copolymers, terpolymers and combinations thereof. In some embodiments, the organic phase biodegradable polymer comprises a poly(D,L-lactide-co-glycolide) having a lactide to glycolide ratio of about 75:25 to about 50:50.

In some embodiments, the cyclodextrin is an alpha cyclodextrin, a beta cyclodextrin, a gamma cyclodextrin, a 2-hydroxypropyl-β-cyclodextrin, a β-cyclodextrin sulfobutylether, hydroxyethyl-β-cyclodextrin, methyl-β-cyclodextrin, dimethyl-β-cyclodextrin, carboxymethyl-β-cyclodextrin, carboxymethyl ethyl-β-cyclodextrin, diethyl-β-cyclodextrin, tri-O-alkyl-1-β-cyclodextrin, glycosyl-β-cyclodextrin, maltosyl-β-cyclodextrin or any derivative thereof, and any combination thereof.

In some embodiments, the microsphere comprises 1%-20% cyclodextrin by weight. In some embodiments, the microsphere comprises 1%-80% carnosine by weight.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

FIG. 1A-C shows a photomicrograph images of the microspheres disclosed herein. FIG. 1A shows a photomicrograph images of the microspheres produced in batch 15 (B15) disclosed herein. FIG. 1B shows a photomicrograph images of the microspheres produced in batch 16 (B16) disclosed herein. FIG. 1C shows a photomicrograph images of the microspheres produced in batch 17 (B17) disclosed herein.

FIG. 2 shows the UV absorption standard curve used to calculate percentage of encapsulation as disclosed herein.

FIG. 3A-D shows the compositions and microsphere characteristics for batches B15, B16, B17, B18, and B19 as disclosed herein. FIG. 3A shows average particle size and distribution of microsphere for the respective batches, as well as the cyclodextrin and polymer compositions used. FIG. 3B shows characteristics of the microspheres for the respective batches, as well as well as the cyclodextrin and polymer compositions used. FIG. 3C is a histogram plot showing the distribution of microsphere particle sizes for the respective batches. FIG. 3D shows a photograph of batches B15, B16, B17, B18, and B19 in glass vials.

FIG. 4A-C shows the compositions and microsphere characteristics for batches B28, B29, B30, B31, and B32 as disclosed herein. FIG. 4A shows average particle size and distribution of microsphere for the respective batches, as well as the cyclodextrin and polymer compositions used. FIG. 4B shows characteristics of the microspheres for the respective batches, as well as well as the cyclodextrin and polymer compositions used. FIG. 4C is a histogram plot showing the distribution of microsphere particle sizes for the respective batches.

FIG. 5A-C shows the compositions and microsphere characteristics for batches B43, B44, and B45 as disclosed herein. FIG. 5A shows average particle size and distribution of microsphere for the respective batches, as well as the cyclodextrin and polymer compositions used.

FIG. 5B shows characteristics of the microspheres for the respective batches, as well as well as the cyclodextrin and polymer compositions used. FIG. 5C is a histogram plot showing the distribution of microsphere particle sizes for the respective batches.

FIG. 6A-C shows the compositions and microsphere characteristics for batches B46, B47, and B48 as disclosed herein. FIG. 6A shows average particle size and distribution of microsphere for the respective batches, as well as the cyclodextrin and polymer compositions used.

FIG. 6B shows characteristics of the microspheres for the respective batches, as well as well as the cyclodextrin and polymer compositions used. FIG. 6C is a histogram plot showing the distribution of microsphere particle sizes for the respective batches.

FIG. 6D shows a graph summarizing release data from the microspheres according to some embodiments disclosed herein. The Y axis show % of S212 release; X axis shows time in days.

FIG. 6E show a table summarizing the release and encapsulation characteristics for scaled-up batched of S212 microsphere for use in animal studies.

FIGS. 7A and 7B shows data of changes in gene expression after treatment of carnosine in forebrain neurons (FIG. 7A) and astrocytes (FIG. 7B).

FIG. 8A to 8D shows raw data of rotarod testing in PD mouse model.

FIG. 9A to 9D shows raw data of pole-climbing testing in PD mouse model.

FIG. 10A to 10C shows raw data of grip strength testing in PD mouse model.

FIG. 11A to 11B shows raw data of bodyweight testing in PD mouse model.

FIG. 12 shows data of the effect of S212 microsphere via subcutaneous injection for 3 times (on Day 1, Day 6 and Day 18) on the latency of the first fall in the rotarod test in MPTP-induced PD model mice (Mean±SEM, n=10).

FIG. 13 shows data of the effect of S212 microsphere via subcutaneous injection for 3 times (on Day 1, Day 6 and Day 18) on the time of pole climbing in MPTP-induced PD model mice (Mean±SEM, n=10).

FIG. 14 shows data of the effect of S212 microsphere via subcutaneous injection for 3 times (on Day 1, Day 6 and Day 18) on the gait score of pole climbing in MPTP-induced PD model mice (Mean±SEM, n=10).

FIG. 15 shows data of the effect of S212 microsphere via subcutaneous injection for 3 times (on Day 1, Day 6 and Day 18) on the Grip strength in MPTP-induced PD model mice (Mean±SEM, n=10).

FIG. 16 shows data of individual and mean plasma concentration-time data of L-Carnosine after intramuscular injection in animal model.

### DETAILED DESCRIPTION

### Definitions

The term “about” is used here in conjunction with numeric values to include normal variations in measurements as expected by persons skilled in the art, and is understood have the same meaning as “approximately” and to cover a typical margin of error, such as +5% of the stated value.

Terms such as “a,” “an,” and “the” are not intended to refer to only a singular entity, but include the general class of which a specific example may be used for illustration.

The terms “a,” “an,” and “the” are used interchangeably with the term “at least one.” The phrases “at least one of” and “comprises at least one of” followed by a list refers to any one of the items in the list and any combination of two or more items in the list.

As used here, the term “or” is generally employed in its usual sense including “and/or” unless the content clearly dictates otherwise. The term “and/or” means one or all of the listed elements or a combination y two or more of the listed elements.

Any amounts (e.g., concentrations) of components in a composition given as a percentage (%) refer to a percentage by weight per volume unless otherwise indicated.

As used herein, “subject” refers to any mammal, including humans, domestic and farm animals, and zoo, sports, or pet animals, such as dogs, horses, cats, sheep, pigs, cows, etc. The preferred subject herein is a human subject, including adults, children, and the elderly.

The terms “treat,” “treated,” or “treating” as used herein refers to therapeutic treatment and/or prophylactic or preventative measures, wherein the object is to prevent or slow down (lessen) an undesired physiological condition, disorder or disease, or to obtain beneficial or desired clinical results. As used herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects.

As used herein, the term a “therapeutically effective amount” is an amount sufficient to effect beneficial or desired results. A therapeutically effective amount can be administered in one or more doses. The therapeutically effective amount is generally determined by a physician on a case-by-case basis and is within the skill of one in the art. Several factors are typically taken into account when determining an appropriate dosage. These factors include age, sex and weight of the patient, the condition being treated, the severity of the condition and the form of the composition being administered.

References to a “neurodegenerative condition”, a “neurodegenerative disorder” or a “neurodegenerative disease”, are used interchangeably, and should be understood as a reference to a condition characterized by neurologically based cognitive, emotional and behavioral disturbances. Neurodegenerative conditions may affect brain or peripheral nerve function. They result from the deterioration of neurons and they are characterized by progressive central or peripheral nervous dysfunction. They are divided into two groups: conditions causing problems with movement or sensation and conditions affecting memory or related to dementia. For example, neurodegenerative conditions in accordance with the invention may include Alzheimer's disease, Alexander disease, Alper's disease, amyotrophic lateral sclerosis, ataxia, telangiectasia, Canavan disease, Cockayne syndrome, corticobasal degeneration, Creutzfeldt-Jakob disease, dementia, Huntington disease, Kennedy's disease, Krabbe disease, Lewy body dementia, Machado-Joseph disease, multiple sclerosis, Parkinson's disease, Pelizaeus-Merzbacher disease, Fronto-Temporal Dementia, Pick's disease, primary lateral sclerosis, Refsum's disease, Sandhoff disease, Schilder's disease, Steele-Richardson-Olszewski disease, tabes dorsalis, Guillain-Barre Syndrome and peripheral neuropathies such as traumatic (nerve severing or crushing), ischemic, metabolic (diabetes, uraemia), infectious, alcoholic, iatrogenic, and genetic neuropathies.

The term “dementia”, as used herein would be understood to persons skilled in the art and includes conditions characterized by neurologically-based cognitive, emotional and behavioral impairment, in particular the Diagnostic and Statistical Manual of Mental Disorders IV outlines characterizes dementia by the presence of: Multiple cognitive deficits, including memory impairment and at least one of the following: aphasia, apraxia, agnosia or disturbance in executive functioning; and impairment of social or occupational function.

Reference to “characteristic symptoms of dementia”, “characteristic symptoms of Alzheimer's disease”, and “characteristic symptoms of Parkinson's disease” should be understood as a reference to any one or more symptoms which may occur in an individual suffering from dementia, in particular dementia associated with disease such as Alzheimer's disease or Parkinson's disease. These symptoms may be evident throughout the disease course or they may be evident only transiently or periodically. For example, an individual may exhibit severe memory impairment impaired social function in response to specific environmental cues or stressors. It should also be understood that the subject symptoms may not necessarily be exhibited by all individuals suffering from Alzheimer's disease or Parkinson's disease. For example, some individuals may suffer from cognitive deficits without obvious impairment of social function. However, for the purpose of the present invention, any such symptoms, irrespective of how many or few patients ever actually exhibit the given symptom, are encompassed by this definition. Without limiting the present invention to any one theory or mode of action, the symptoms that are most commonly associated with Alzheimer's disease include cognitive deficits and impaired social or occupational function, and the symptoms that are most commonly associated with Parkinson's disease include impaired gross and fine motor functions. For certain of the abovementioned conditions it is clear that the methods of the invention may be used prophylactically as well as for the alleviation of acute symptoms.

As used herein, the term “lyoprotection” refers to stabilization during all of the freeze-drying process (i.e., during both freezing and drying). Such stabilization is often required for freeze-drying of materials such as proteins, peptides and biological drugs. This is because complex molecules often require a moderate level of residual water to maintain structure and function. Accordingly, a “lyoprotectant” protects the structure and/or function of composition during lyophilization (e.g., prevents aggregation, improves bioavailability, increases stability and/or improves membrane integrity and cargo retention).

As used herein, the term “lyophilization” (also known as “lyophilizing,” “freeze drying” or “cryodessication”) refers to a low temperature dehydration process that involves freezing a product and lowering pressure, removing the ice by sublimation. Lyophilizing may comprise freezing the composition at a temperature of greater than −40° C., or e.g. 55 less than −30° C., forming a frozen composition; and drying the frozen composition to form the lyophilized composition. The step of drying may occur at 50 mTorr at a temperature of −25 to −34° C., or −30 to −34° C.

As used herein, the term “entrapped” or “encapsulated” by a microsphere polymer with respect to some composition disclosed herein (e.g., a peptide) means that the composition is embedded within any part of a structure of the microsphere polymer. The composition may be located in any part of the structure, such as within an inner cavity of the structure or within pores of the structure. The term “entrapped” by a cyclodextrin with respect to some composition disclosed herein (e.g., a peptide) means that the composition forms and inclusion complex with the cyclodextrin.

### Microsphere Formulations

According to some aspects, the present disclosure provides a microsphere polymer delivery system effective for sustained localized and/or systemic delivery of a therapeutic peptide in a subject. The affinity between peptides and microsphere polymers can vary based on their respective chemical properties. For example, during the production process of microspheres hydrophilic drugs may partition into the external aqueous phase during fabrication, reducing encapsulation. Furthermore, peptide/microsphere polymer interactions may negatively impact stability of primary and/or secondary emulsions, which can lead to peptide leakage before microspheres harden.

Here is has been surprisingly discovered that that combining a microsphere polymer with a cyclodextrin during microsphere production for delivery of peptides can significantly improve one or more of encapsulation efficiency and release profile. In some embodiments, combining cyclodextrin with microsphere polymer (e.g. PLGA) in an organic phase used in an emulsion preparation with peptide in an aqueous phase is effective to improve the encapsulation efficiency and release profile of the peptide from the resulting microsphere. In some embodiments, the cyclodextrin is able to draw the peptide (e.g., via formation of inclusion complex) from the aqueous phase into the organic phase to produce a unique distribution of peptide in the final microsphere product.

According to some aspects, the present disclosure provides a drug delivery system effective for sustained localized and/or systemic delivery of a therapeutic agent in a subject. In some embodiments, the drug delivery system is effective for the treatment of a neurodegenerative condition and comprises a plurality of biodegradable microspheres. In some embodiments, the biodegradable microspheres comprise a biodegradable polymer matrix and a therapeutic agent effective for the treatment of the neurodegenerative condition. In some embodiments, the therapeutic agent is dispersed within and/or encapsulated by the polymer matrix (i.e. entrapped). In some embodiments, the therapeutic agent-containing microspheres can be produced by an emulsion process. In some embodiments, the therapeutic agent-containing microspheres can be selected for microspheres of particular sizes.

In some embodiments, the microspheres disclosed herein comprise a hydrogel, such as cyclodextrin. In some embodiments, the hydrogel is complexed with the therapeutic agent and dispersed within and/or encapsulated by the polymer matrix of the biodegradable polymer.

In some embodiments, the microspheres disclosed herein have diameters of about 1 μm to about 150 μm. In some embodiments, the microspheres disclosed herein have diameters of about 10 μm to about 50 μm. In some embodiments, the microspheres disclosed herein have diameters of about 10 μm to 30 μm. In some embodiments, the microspheres disclosed herein have diameters of about 30 μm to about 50 μm.

In some embodiments, the therapeutic agent-containing microspheres disclosed herein may be characterized by mean diameter (or mean particle size) and/or particle size distribution of a population of microspheres. For example, measures that may be derived from the microsphere size distribution include the mean and modal diameter. The d90 and d10 are the diameters below which 90% and 10% of the particles fall. The value d50 is the median diameter, or diameter below which 50% of the particles fall, and d75 and d25 are the diameters below which 75% and 25% of the particles fall, respectively. The size and distribution of a microsphere population can be measured using laser light scattering methods. Instruments suitable for measuring the size and distribution of a microsphere population are commercially available, from, for example, Malvern Instruments Ltd. Microspheres containing a therapeutic agent, such as those produced by emulsion methods, may be spherical or substantially spherical. In these instances, the particle size(s) measured and reported by such instruments for a sample of microspheres will essentially represent the diameter(s) of the microspheres. The size and distribution of a microsphere population can also be measured manually using light microscopy and size metrics (e.g. micron ruler).

In some embodiments, the microspheres disclosed herein have a mean diameter of about 19.8 μm, 12 μm, 29.2 μm, 6.6 μm, 6.4 μm, 70.8 μm, 65.5 μm, 38 μm, 51.7 μm, 28.1 μm, 1.2 μm, 57 μm, 68.8 μm, 13.86 μm, 14.43 μm, or 27.71 μm. In some embodiments, the microspheres disclosed herein have a mean diameter of no less than 1 μm and no greater than 71 μm. In some embodiments, the microspheres disclosed herein have a mean diameter of no less than 10 μm and no greater than 50 μm. In some embodiments, the microspheres disclosed herein have a mean diameter of no less than 10 μm and no greater than 30 μm. In some embodiments, the microspheres disclosed herein have a mean diameter of no less than 30 μm and no greater than 50 μm.

In some embodiments, the span (d90-d10) of the microspheres disclosed herein may be no more than about 30 μm, 100 μm, or 70 μm.

In some embodiment, the microspheres disclosed herein have a unimodal particle size distribution and the diameters of microspheres deviate from the modal diameter by no more than about 20 μm, 30 μm, 40 μm, 50 μm, 60 μm, or 70 μm.

According to some aspects, the present disclosure provides a therapeutic agent containing microsphere effective for the treatment of a neurological disease or condition, such as Alzheimer's disease or Parkinson's disease.

In some embodiments, the microspheres may comprise from about 5% to about 80% by weight of a therapeutic agent. In some embodiments, the microspheres may comprise from about 5% to about 80% by weight of carnosine. In some embodiments the microspheres comprise from about 5% to about 40% by weight of a therapeutic agent. In some embodiments the microspheres comprise from about 5% to about 40% by weight of carnosine.

In some embodiments, the microspheres comprise a biodegradable polymer, a therapeutic agent, and optionally a pharmaceutically acceptable excipient. In some embodiments, the biodegradable polymer comprises poly(D,L-lactide), a poly(D,L-lactide-co-glycolide), a poly(ortho ester), a poly(phosphazine), a poly(phosphate ester), a polycaprolactone, a polyethylene glycol, sodium-gamma-polyglutamate, hyaluornic acid (various molecular weights) a naturally occurring polymer, or any combination thereof. In some embodiments, naturally occurring polymers include gelatin and collagen. In some embodiments, microspheres may comprise both a poly(D,L-lactide) and a poly(D,L-lactide-co-glycolide).

In some embodiments, the microspheres disclosed herein comprise one or more of polyvinyl alcohol, polyanhydrides, polyamines, polyesteramides, polydioxanones, polyacetals, polyketals, polycarbonates, polyphosphesters, polyethers, polyesters, polybutylene, terephthalate, polyorthocarbonates, polyphosphazenes, succinates, poly(malic acid), poly(amino acids), polyvinypyrrolidone, polysaccharides, oligosaccharides, alginate copolymers, terpolymers, and combinations thereof that are biocompatible and may be biodegradable.

The biodegradable polymeric materials which are included to form the microsphere may be subject to enzymatic or hydrolytic instability. Water soluble polymers may be cross-linked with hydrolytic or biodegradable unstable cross-links to provide useful water insoluble polymers. The degree of stability can be varied widely, depending upon the choice of monomer, whether a homopolymer or copolymer is employed, employing mixtures of polymers, and whether the polymer includes terminal acid groups.

Poly(lactide-co-glycolide) or PLGA, includes poly(D,L-lactide-co-glycolide), also identified by CAS Number 26780-50-7, and may be represented by a formula:

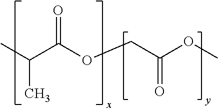

Where x and y=the number of repeating units. Thus, poly(D,L-lactide-co-glycolide) comprises one or more blocks of D,L-lactide repeat units and one or more blocks of glycolide repeat units, where the size and number of the respective blocks may vary.

The molar percent of each monomer (repeat unit) in a poly(lactide-co-glycolide) (PLGA) copolymer may be 0-100%, 15-85%, about 25-75%, or about 35-65%. In some embodiments, the D,L-lactide to glycolide ratio may be about 50:50 or about 75:25. The PLA and/or PLGA polymer included in the microsphere may comprise ester or free carboxylic acid end groups. PLA and PLGA polymers are available commercially, e.g., from Evonik Industries AG, Germany, under the RESOMER® product line.

In some embodiments, the microsphere disclosed herein comprise a cyclic oligosaccharide-based polymer. In some embodiments, the cyclic oligosaccharide-based polymer comprises cyclodextrin. In some embodiments, the cyclic oligosaccharide-based polymer comprises alpha, beta, or gamma cyclodextrin or combinations thereof. In some embodiments, the cyclodextrin comprises non-crosslinked cyclodextrin. In some embodiments, the cyclodextrin comprises crosslinked cyclodextrin.

In some embodiments, the cyclic oligosaccharide-based polymer comprises an alpha cyclodextrin, beta cyclodextrin, a gamma cyclodextrin, a 2-hydroxypropyl-β-cyclodextrin, a β-cyclodextrin sulfobutylether, hydroxyethyl-β-cyclodextrin, methyl-β-cyclodextrin, dimethyl-β-cyclodextrin, carboxymethyl-β-cyclodextrin, carboxymethyl ethyl-β-cyclodextrin, diethyl-β-cyclodextrin, tri-O-alkyl-1-β-cyclodextrin, glycosyl-β-cyclodextrin, maltosyl-β-cyclodextrin or any derivative thereof, and any combination thereof. In some embodiments, the cyclic oligosaccharide-based polymer is an alkylated derivative. In some embodiments, the microsphere composition comprises, by weight of the total composition, 0.1%-20% (0.1, 0.5, 0.7, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20% or any value therebetween) cyclic oligosaccharide-based polymer. In some embodiments, the microsphere composition comprises, by weight of the total composition, 0.1%-20% (0.1, 0.5, 0.7, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20% or any value therebetween) cyclodextrin or an alkylated derivative thereof. In some embodiments, the microsphere composition comprises, by weight of the total composition, 0.1%-20% (0.1, 0.5, 0.7, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20% or any value therebetween) gamma cyclodextrin or an alkylated derivative thereof.

The amount of cyclodextrin used in the compositions and methods disclosed herein varies according to the physiochemical properties, pharmacokinetic properties, side effect or adverse events, composition considerations, or other factors associated with the therapeutically active agent, or a salt or prodrug thereof, or with the properties of other excipients in the composition. In some embodiments, the concentration or amount of cyclodextrin used in accordance with the compositions and methods disclosed herein will vary, depending on the need. When used, the amount of cyclodextrins needed to increase solubility of a therapeutic agent and/or function as a controlled-release excipient in any of the compositions described herein is selected using the principles, examples, and teachings described herein.

In some embodiments, the alpha cyclodextrin has the following chemical formula:

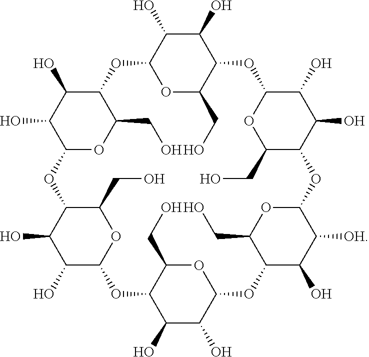 [SMILES: OC[C@H]1O[C@@H]2O[C@H]3[C@H](O)[C@@H](O)[C@@H](O[C@H]4[C@H](O)[C@@H](O)[C@@H](O[C@H]5[C@H](O)[C@@H](O)[C@@H](O[C@H]6[C@H](O)[C@@H](O)[C@@H](O[C@H]7[C@H](O)[C@@H](O)[C@@H](O[C@H]1[C@H](O)[C@H]2O)O[C@@H]7CO)O[C@@H]6CO)O[C@@H]5CO)O[C@@H]4CO)O[C@@H]3CO]

In some embodiments, the beta cyclodextrin has the following chemical formula:

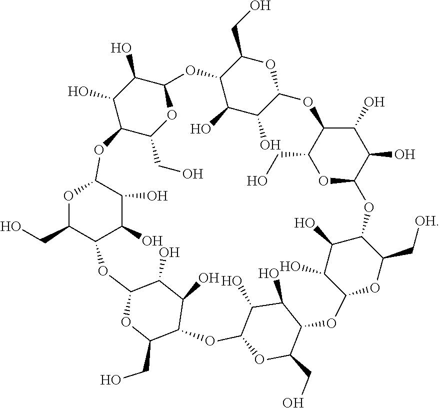 [SMILES: OC[C@H]1O[C@@H]2O[C@H]3[C@H](O)[C@@H](O)[C@@H](O[C@H]4[C@H](O)[C@@H](O)[C@@H](O[C@H]5[C@H](O)[C@@H](O)[C@@H](O[C@H]6[C@H](O)[C@@H](O)[C@@H](O[C@H]7[C@H](O)[C@@H](O)[C@@H](O[C@H]8[C@H](O)[C@@H](O)[C@@H](O[C@H]1[C@H](O)[C@H]2O)O[C@@H]8CO)O[C@@H]7CO)O[C@@H]6CO)O[C@@H]5CO)O[C@@H]4CO)O[C@@H]3CO]

In some embodiments, the gamma cyclodextrin has the following chemical formula:

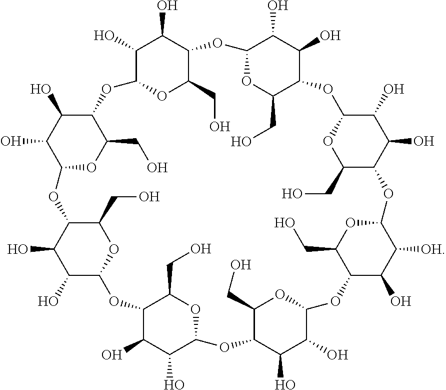 [SMILES: OC[C@H]1O[C@@H]2O[C@H]3[C@H](O)[C@@H](O)[C@@H](O[C@H]4[C@H](O)[C@@H](O)[C@@H](O[C@H]5[C@H](O)[C@@H](O)[C@@H](O[C@H]6[C@H](O)[C@@H](O)[C@@H](O[C@H]7[C@H](O)[C@@H](O)[C@@H](O[C@H]8[C@H](O)[C@@H](O)[C@@H](O[C@H]9[C@H](O)[C@@H](O)[C@@H](O[C@H]1[C@H](O)[C@H]2O)O[C@@H]9CO)O[C@@H]8CO)O[C@@H]7CO)O[C@@H]6CO)O[C@@H]5CO)O[C@@H]4CO)O[C@@H]3CO]

In some embodiments, the cyclodextrin may comprise one or more substituted positions of the hydroxyl groups. In some embodiments the degree of substitution of the cyclodextrin is between 3 and 8. In some embodiments the degree of substitution of the cyclodextrin is between 3.5 and 4.9. In some embodiments the degree of substitution of the cyclodextrin is between 4.1 and 5.1. In some embodiments the degree of substitution of the cyclodextrin is between 6 and 8.

In some embodiments, the microspheres disclosed herein comprise an antioxidant. In some embodiments, the antioxidant comprises carnosine. In some embodiments, the antioxidant comprises L-carnosine, D-carnosine, acetyl-carnosine, anserine, alanine, L-histidine, D-histidine, a derivative thereof, or a combination thereof. In some embodiments, the antioxidant comprises anserine. In some embodiments, the antioxidant comprises L-carnosine. In some embodiments, the intranasal composition comprises, by weight of the total composition, 0.5%-80% (0.5, 0.7, 1, 1.5, 2, 2.5, 3, 3.5, 4, 4.5, 5, 5.5, 6, 6.5, 7, 7.5, 8, 8.5, 9, 9.5, 10, 20, 30, 40, 50, 60 or 70% or any value therebetween) antioxidant. In some embodiments, the microsphere composition comprises, by weight of the total composition, 0.5%-80% (0.5, 0.7, 1, 1.5, 2, 2.5, 3, 3.5, 4, 4.5, 5, 5.5, 6, 6.5, 7, 7.5, 8, 8.5, 9, 9.5, 10, 20, 30, 40, 50, 60 or 70% or any value therebetween) carnosine. In some embodiments, the intranasal composition comprises, by weight of the total composition, 0.5%-80% (0.5, 0.7, 1, 1.5, 2, 2.5, 3, 3.5, 4, 4.5, 5, 5.5, 6, 6.5, 7, 7.5, 8, 8.5, 9, 9.5, 10, 20, 30, 40, 50, 60 or 70% or any value therebetween) L-carnosine.

In some embodiments, a drug delivery system according to the present disclosure comprises a plurality of microspheres and a pharmaceutically acceptable carrier. Pharmaceutically acceptable carriers are carriers (e.g., liquids, oils, or gels) that are biocompatible with mammalian tissue. In some embodiments, two or more such carriers may be included in the drug delivery system. In some embodiments, the microspheres may be suspended in the carrier. In some embodiments, the drug delivery system can be in the form of a gel or suspension. Examples of pharmaceutically acceptable carriers include, but may not be limited to, sterile water; aqueous solutions comprising one or more buffering agents and having a pH of about 4 to about 8, or about 7.0 to about 7.8); and aqueous solutions (or gels) comprising hyaluronic acid (or an alkali or alkaline earth metal salt of hyaluronic acid such as sodium hyaluronate), hydroxyethyl cellulose (HEC), carboxymethylcellulose (CMC), hydrocypropylmethyl cellulose (HPMC), polyvinylproline (PVP), or a pluronic polymer.

According to some aspects, the microsphere composition disclosed herein may further comprise one or more pharmaceutically acceptable excipients, such as one or more buffering agents, tonicity agents, preservatives, or polyethylene glycols. Suitable buffering agents include, without limitation, alkali and alkaline earth carbonates, phosphates, bicarbonates, citrates, borates, acetates, succinates and the like, such as sodium phosphate, citrate, borate, acetate, bicarbonate, carbonate and the like. These agents are advantageously present in amounts sufficient to maintain a pH of the system of between 2 and 9 and more preferably between about 4 and about 8.

In some embodiments, the microsphere composition comprises a preservative. As used herein the term “preservative” refers to any known pharmaceutically acceptable preservative that functions by inhibiting bacteria, fungi, yeast, mold, other microbe. Suitable preservatives include but are not limited to antimicrobial agents. In some embodiments, antimicrobial agents comprise potassium sorbate, sodium benzoate, paraben, benzyl alcohol, sorbic acid, triclosan, phenoxyisopropanol, diazolidinyl urea, bronopol, Alkyl (C12-22) trimethyl ammonium bromide, Alkyl (C12-22) trimethyl ammonium chloride, Benzalkonium chloride, Benzalkonium bromide, Benzalkonium saccharinate, ethylhexylglycerin, phenoxyethanol, or a combination thereof. Suitable preservatives may also include sodium bisulfite, sodium bisulfate, sodium thiosulfate, ascorbate, benzalkonium chloride, chlorobutanol, thimerosal, phenylmercuric acetate, phenylmercuric borate, phenylmercuric nitrate, parabens, methylparaben, polyvinyl alcohol, benzyl alcohol, phenylethanol and the like and mixtures thereof. Useful tonicity agents include glycerin, sugar alcohols, xylitol, sorbitol, glycerol, erythritol, mannitol, salts, potassium chloride and/or sodium chloride. Useful polyethylene glycols have a molecular weight of about 300 to about 40,000. Specific examples of polyethylene glycols that may be included in a microsphere formulation include polyethylene glycol 3350 (PEG 3350), PEG 4400, and PEG 8000. In other instances, a polyethylene glycol with a molecular weight of about 20,000 (PEG 20K) may be used. Where the microsphere compositions disclosed herein comprise a preservative, the preservative may be selected from any pharmaceutically acceptable preservative. In still other embodiments, the microsphere compositions disclosed herein do not contain a preservative.

In some embodiments, the microsphere composition comprises, by weight of the total composition 0.05%-0.5% (such as 0.1, 0.12, 0.13, 0.14, 0.15% or any value therebetween) preservative. In some embodiments, the microsphere composition comprises, by weight of the total composition 0.05%-0.5% (such as 0.1, 0.12, 0.13, 0.14, 0.15% or any value therebetween) potassium sorbate.

According to some aspects, the present disclosure provides a microsphere composition for therapeutic treatment of neurodegenerative diseases or disorders. In other embodiments, the present disclosure provides microsphere compositions for prophylactic treatment of neurodegenerative diseases or disorders. It has been surprisingly found that delivery of an antioxidant, such as carnosine, in microsphere form can be produced in combination with cyclodextrin to provide a long acting release of carnosine to a neurodegenerative disease. In some embodiments, microsphere delivery of a compositions comprising carnosine, cyclodextrin, and a polymer may provide sustained release of carnosine locally or systemically.

The formulations as disclosed herein may allow an active agent, such as carnosine, to be absorbed in a sustained manner providing improved bioavailability at low or reduced doses and/or longer duration of action. In some embodiments, the formulations of the present invention may also provide a reduced incidence of side effects when compared with other drug delivery methods.

In some embodiments, the microspheres disclosed herein comprise one or more of:

1%-80% carnosine
1%-80% cyclodextrin
1%-80% PVA
1%-80% PLGA

In some embodiments, the composition disclosed herein comprises one or more of:

1-40% carnosine
1-20% cyclodextrin
1-30% PVA
1-8% Polyvinyl alcohol
0.1-1% Sodium hyaluronate

In some embodiments, the composition disclosed herein comprises one or more of:

1-50% Carnosine
1-30% cyclodextrin
1-20% PLGA
1-10% Sodium Polyglutamate (γ-PGA)

In some embodiments, the composition disclosed herein comprises one or more of:

1-20% carnosine
1-50% cyclodextrin
1-20% Sodium Polyglutamate (γ-PGA)
1-50% PLGA
1-15% Polyvinyl alcohol

In some embodiments, the microsphere composition comprises a chelator, such as EDTA. In some embodiments, the microsphere composition comprises, by weight of the total composition, 0.001%-0.05% (such as 0.005, 0.01, 0.015, 0.2% or any value therebetween) chelator. In some embodiments, the microsphere composition comprises, by weight of the total composition 0.001%-0.05% (such as 0.005, 0.01, 0.015, 0.2% or any value therebetween) EDTA.

In some embodiments, the intranasal composition disclosed herein comprises a pH modifying agent. In some embodiments, the composition has a pH between 7.5 and 9.0 (e.g., pH 8.0, 8.1, 8.2, 8.3, 8.4, 8.5 or any value therebetween). In some embodiments, the composition has a pH between 8.25 and 8.31 (e.g., pH 8.25, 8.26, 8.27, 8.28, 8.29, 8.3, 8.31, or any value therebetween).

### Lyophilization

In some embodiments, the formulations disclosed herein are lyophilized. In some embodiments, lyophilized formulations comprise compositions that can protect the integrity and/or structure of the formulation (i.e. a lyoprotectant). In some embodiments, the lyoprotectant is as disclosed in U.S. Pat. No. 11,833,224, which is incorporated herein by reference in its entirety. In some embodiments, formulations are lyophilized using the compositions of the instant disclosure to retain at least 90%, 91%, 92%, 93%, 94%, 95%, 96%, 97%, 98%, 99% or more of their biological activity and/or integrity when reconstituted/thawed. In a specific embodiment, the compositions of the instant disclosure are effective at protecting biological activity and/or structural integrity of microsphere particles at least 90%, 91%, 92%, 93%, 94%, 95%, 96%, 97%, 98%, 99% or more as compared to microsphere particles that have not been lyophilized.

In some embodiments, the lyoprotectant composition comprises 0.3%-8% (e.g., 0.3, 0.5, 0.7, 0.9, 1, 1.2, 1.5, 1.7, 2, 2.2, 2.5, 2.7, 3, 3.2, 3.5, 3.7, 4, 4.2, 4.5, 4.7, 5, 5.2, 5.5, 5.7, 6, 6.2, 6.5, 6.7, 7, 7.2, 7.5, 7.7, 8%, or any value therebetween) cyclic oligosaccharide-based polymer, 2%-10% (e.g., 2, 2.2, 2.5, 2.7, 3, 3.2, 3.5, 3.7, 4, 4.2, 4.5, 4.7, 5, 5.2, 5.5, 5.7, 6, 6.2, 6.5, 6.7, 7, 7.2, 7.5, 7.7, or 8, 8.2, 8.5, 8.7, 9, 9.2, 9.5, 9.7, 9.9, 10%, or any value therebetween) sugar, and 0.2%-10% (e.g., 0.2, 0.3, 0.5, 0.7, 0.9, 1, 1.2, 1.5, 1.7, 2, 2.2, 2.5, 2.7, 3, 3.2, 3.5, 3.7, 4, 4.2, 4.5, 4.7, 5, 5.2, 5.5, 5.7, 6, 6.2, 6.5, 6.7, 7, 7.2, 7.5, 7.7, 8, 8.2, 8.5, 8.7, 9, 9.2, 9.5, 9.7, 9.9, 10%, or any value therebetween) amino acid.

In some embodiments, the lyoprotectant composition comprises 1%-5% (e.g., 1, 1.2, 1.5, 1.7, 2, 2.2, 2.5, 2.7, 3, 3.2, 3.5, 3.7, 4, 4.2, 4.5, 4.7, 5, or any value therebetween) cyclic oligosaccharide-based polymer. In some embodiments, the lyoprotectant composition comprises 1%-3% (e.g., 1, 1.2, 1.5, 1.7, 2, 2.2, 2.5, 2.7, 3, or any value therebetween) cyclic oligosaccharide-based polymer. In some embodiments, the composition comprises 2% cyclic oligosaccharide-based polymer.

In some embodiments, the lyoprotectant composition comprises 4%-8% (e.g., 4, 4.2, 4.5, 4.7, 5, 5.2, 5.5, 5.7, 6, 6.2, 6.5, 6.7, 7, 7.2, 7.5, 7.7, 8%, or any value therebetween) sugar. In some embodiments, the lyoprotectant composition comprises 5%-7% (e.g., 5, 5.2, 5.5, 5.7, 6, 6.2, 6.5, 6.7, 7, or any value therebetween) sugar. In some embodiments, the composition comprises 6% sugar.

In some embodiments, the lyoprotectant composition comprises 0.2%-4% (e.g., 0.2, 0.3, 0.5, 0.7, 0.9, 1, 1.2, 1.5, 1.7, 2, 2.2, 2.5, 2.7, 3, 3.2, 3.5, 3.7, 4, or any value therebetween) amino acid. In some embodiments, the lyoprotectant composition comprises 0.3%-0.5% (e.g., 0.3, 0.4 or 0.5%) amino acid. In some embodiments, the lyoprotectant composition comprises 0.5% amino acid.

In a specific embodiment, the lyoprotectant composition comprises about 6% sucrose, about 2% gamma cyclodextrin and about 0.5% trimethylglycine. In a specific embodiment, the lyoprotectant composition comprises about 6% sucrose, about 1% gamma cyclodextrin and about 1% trimethylglycine. In some embodiments, the lyoprotectant composition ingredients are constituted in water or phosphate buffered saline (PBS). In some embodiments, the lyoprotectant is sterilized, optionally by filtering through a 0.2 micron filter.

In some embodiments the compositions disclosed herein comprise 0.3%-8% (e.g., 0.3, 0.5, 0.7, 0.9, 1, 1.2, 1.5, 1.7, 2, 2.2, 2.5, 2.7, 3, 3.2, 3.5, 3.7, 4, 4.2, 4.5, 4.7, 5, 5.2, 5.5, 5.7, 6, 6.2, 6.5, 6.7, 7, 7.2, 7.5, 7.7, 8%, or any value therebetween) cyclic oligosaccharide-based polymer, 2%-10% (e.g., 2, 2.2, 2.5, 2.7, 3, 3.2, 3.5, 3.7, 4, 4.2, 4.5, 4.7, 5, 5.2, 5.5, 5.7, 6, 6.2, 6.5, 6.7, 7, 7.2, 7.5, 7.7, 8, 8.2, 8.5, 8.7, 9, 9.2, 9.5, 9.7, 9.9, 10%, or any value therebetween) sugar, 0.2%-10% (e.g., 0.2, 0.3, 0.5, 0.7, 0.9, 1, 1.2, 1.5, 1.7, 25 2, 2.2, 2.5, 2.7, 3, 3.2, 3.5, 3.7, 4, 4.2, 4.5, 4.7, 5, 5.2, 5.5, 5.7, 6, 6.2, 6.5, 6.7, 7, 7.2, 7.5, 7.7, 8, 8.2, 8.5, 8.7, 9, 9.2, 9.5, 9.7, 9.9, 10%, or any value therebetween) amino acid, and a microsphere particle.

In some embodiments, the composition comprises 1%-5% (e.g., 1, 1.2, 1.5, 1.7, 2, 2.2, 2.5, 2.7, 3, 3.2, 3.5, 3.7, 4, 4.2, 4.5, 4.7, 5, or any value therebetween) cyclic oligosaccharide-based polymer. In some embodiments, the composition comprises 1%-3% (e.g., 1, 1.2, 1.5, 1.7, 2, 2.2, 2.5, 2.7, 3, or any value therebetween) cyclic oligosaccharide-based polymer. In some embodiments, the composition comprises 2% cyclic oligosaccharide-based polymer.

In some embodiments, the composition comprises 4%-8% (e.g., 4, 4.2, 4.5, 4.7, 5, 5.2, 5.5, 5.7, 6, 6.2, 6.5, 6.7, 7, 7.2, 7.5, 7.7, 8%, or any value therebetween) sugar. In some embodiments, the composition comprises 5%-7% (e.g., 5, 5.2, 5.5, 5.7, 6, 6.2, 6.5, 6.7, 7, or any value therebetween) sugar. In some embodiments, the composition comprises 6% sugar.

In some embodiments, the composition comprises 0.2%-4% (e.g., 0.2, 0.3, 0.5, 0.7, 0.9, 1, 1.2, 1.5, 1.7, 2, 2.2, 2.5, 2.7, 3, 3.2, 3.5, 3.7, 4, or any value therebetween) amino acid. In some embodiments, the composition comprises 0.3%-0.5% (e.g., 0.3, 0.4 or 0.5%) amino acid. In some embodiments, the composition comprises 0.5% amino acid. In a specific embodiment, the composition comprises about 6% sucrose, about 2% gamma cyclodextrin and about 0.5% trimethylglycine.

In some embodiments, the cyclic oligosaccharide-based polymer comprises an alpha cyclodextrin, beta cyclodextrin, a gamma cyclodextrin, a 2-hydroxypropyl-β-cyclodextrin, a β-cyclodextrin sulfobutylether, hydroxyethyl-β-cyclodextrin, methyl-β-cyclodextrin, dimethyl-β-cyclodextrin, carboxymethyl-β-cyclodextrin, carboxymethyl ethyl-β-cyclodextrin, diethyl-β-cyclodextrin, tri-O-alkyl-1β-cyclodextrin, glocosyl-β-cyclodextrin, maltosyl-β-cyclodextrin or any derivative thereof, and any combination thereof. In some embodiments, the cyclic oligosaccharide-based polymer comprises gamma cyclodextrin. In some embodiments, the alpha cyclodextrin has the chemical formula as disclosed herein. In some embodiments, the beta cyclodextrin has the chemical formula as disclosed herein. In some embodiments, the gamma cyclodextrin has the chemical formula as disclosed herein. In some embodiments, the sugar comprises sucrose, mannitol, and/or trehalose. In some embodiments, the sugar comprises sucrose. In some embodiments, the amino acid comprises trimethylglycine, glycine, arginine or any salts thereof.

In some embodiments, the microsphere particle stabilized using the lyoprotectant composition of the instant disclosure during lyophilization retains at least 90% (e.g., at least 90%, 91%, 92%, 93%, 94%, 95%, 96%, 97%, 98%, 99% or more) of its structural integrity when resolubilized. In some embodiments, the lyoprotectant composition reduces agglomeration and aggregation of the biologic drug during lyophilization by at least 90% (e.g., at least 90%, 91%, 92%, 93%, 94%, 95%, 96%, 97%, 98%, 99% or more). In some embodiments, the microsphere stabilized using the lyoprotectant composition during lyophilization retains the bioavailability by at least 90% (e.g., at least 90%, 91%, 92%, 93%, 94%, 95%, 96%, 97%, 98%, 99% or more) when resolubilized.

In some embodiments, the microsphere particle is stabilized using the lyoprotectant composition of the instant disclosure during lyophilization, and the lyophilized composition comprising the microsphere particle when resolubilized has a zeta potential that is lower than −25 mV, or lower than −26 mV, or lower than −27 mV, or lower than −28 mV, or lower than −29 mV, or lower than −30 mV, or lower than −31 mV, or lower than −32 mV, or lower than −33 my, or lower than −34 mV, or lower than −35 mV, or lower than −36 mV, or lower than −37 mV, or lower than −38 mV, or lower than −39 mV, or lower than −40 mV.

In some embodiments, the microsphere particle is stabilized using the lyoprotectant composition of the instant disclosure during lyophilization, and the lyophilized composition comprising the microsphere particle when resolubilized has a zeta potential that is no more than 10 mV, no more than 9 mV, no more than 8 mV, no more than 7 mV, no more than 6 mV, no more than 5 mV, no more than 4 mV, no more than 3 mV, no more than 2 mV, or no more than 1 mV, higher than the zeta potential of a corresponding non-lyophilized composition comprising the microsphere particle.

According to some embodiments, the present disclosure provides a method for stabilizing a microsphere particle during lyophilization comprising mixing the microsphere particle with a lyoprotectant blend disclosed herein.

### Methods of Making Microspheres

According to some aspects, the present disclosure provides a method of making microspheres as disclosed herein using an emulsion process. In some embodiments, the method of making microspheres as disclosed herein comprises the steps of: 1) dissolving one or more biodegradable polymers and therapeutic agents in a solvent to form a first solution; 2) dissolving one or more biodegradable polymers in an organic solvent to form a second solution; 3) adding the second solution dropwise to the first solution with stirring to produce a first emulsion; 4) transfer the first emulsion to a separate solution of biodegradable polymer with stirring and allow to stir for a time sufficient to evaporate the organic solvent. According to some embodiments, the microspheres may be harvested with or without filtering to select for a desired particle size. In some embodiments, the microspheres may be harvested by gravity sedimentation and/or centrifugation sedimentation. In some embodiments, microspheres may be harvested by screening mesh. In some embodiments, the harvested microspheres may be lyophilized.

In some embodiments, carnosine is protonated by dissolving the carnosine with an acid preferably an alpha hydroxyl acid or beta hydroxyl acid such as lactic acid or salicylic acid, using either a 1:1 or 0.5:1 molar ratio in polyvinyl alcohol (PVA) solution.

In some embodiments, the microspheres as disclosed herein are produced by the process of: 1) dissolving carnosine in a polyvinyl alcohol (PVA) solution; 2) adding dropwise to the carnosine/PVA a solution of PLGA and cyclodextrin in the organic solvent DCM, followed by stirring to produce a first emulsion; 3) providing a separate solution of PVA and cyclodextrin; 4) transferring the first emulsion to the separate solution of PVA and cyclodextrin with stirring; 5) stir the product of step 4 to allow DCM to evaporate away, thereby producing double emulsion microspheres.

In some embodiments, PLGA polymer with cyclodextrin and sodium gamma polyglutamate is added to dichloromethane (DCM) solution and added dropwise to the first solution. In some embodiments, the second solution contain 2% PVA with or without cyclodextrin and sodium polyglutamate. In some embodiments, first is mixed with this blend for 72 hours.

In some embodiments, polymers can be substituted together or individually in compositions in the ranges of same molar ratio of PLGA or half molar ratio.

In some embodiments, the organic solvent used in the emulsion process is a non-polar solvent, such as chloroform or ethyl acetate.

In some embodiments, the microspheres produced comprise greater than 20%, 30%, 40%, 50%, 60%, 70%, 80%, 85%, 90%, or 95% encapsulation of the initial amount of therapeutic agent.

In some embodiments the microspheres may include two or more therapeutic agents that may each be effective in treating a neurodegenerative condition. In some embodiments, the microspheres comprise a single therapeutic agent.

In some embodiments, biodegradable polymer disclosed herein (such as PLGA) is designed to degrade at a rate effective to sustain release of an amount of the therapeutic agent for a time greater than about one week. In some embodiments, the biodegradable polymer may provide continuous release of the therapeutic agent for about one month (30 days), two months, three months, or 6 months or more from the time the microspheres are administered to a subject.

In some embodiments, the biodegradable polymer may comprise one or more biodegradable polymers. For example, in some embodiments, the microspheres comprise a first and second polymers that differ one from the other with regard to their end groups, inherent viscosity, and/or repeating units. In other embodiments, the microspheres may comprise first, second, and third biodegradable polymers. One or more of the biodegradable polymers may have terminal acid groups. For example, the microspheres may comprise a first biodegradable polymer having an ester end group and a different second biodegradable polymer having an acid end group. Useful biodegradable polymers which may be used independently or in combination include poly(D,L-lactide) polymers and poly(D,L-lactide-co-glycolide) copolymers. In addition, the microspheres may further comprise a polyethylene glycol (PEG). Useful polyethylene glycols include PEG 3350, PEG 4400, and PEG 8000.

In some embodiments, the microspheres disclosed herein have an active agent or agents homogenously distributed throughout the polymeric matrix of the polymer. In some embodiments, the microspheres disclosed herein have an active agent or agents encapsulated, where a reservoir of active agent is encapsulated by the polymer. In some embodiments, the therapeutic agent may be distributed in a non-homogenous pattern in the polymer matrix. For example, the microspheres may include a portion that has a greater concentration of the therapeutic agent relative to a second portion of the microspheres.

In some embodiments, the amount of therapeutic agent loaded into the microspheres will vary widely depending on the effective dosage required and the desired rate of release from the microspheres. In some embodiments, the therapeutic agent will be between about 2 and 40% by weight of the microsphere. In some embodiments, the therapeutic agent constitutes at least about 5%, at least about 10% of the microsphere, or is about 25% of the microspheres. In some embodiments the microspheres comprise between about 5 and about 50% by weight therapeutic agent, or more specifically between about 5 and 30% by weight therapeutic agent.

In some embodiments, the amount of cyclodextrin loaded into the microspheres will vary widely depending on the desired rate of release from the microspheres. In some embodiments, the cyclodextrin will be between about 2 and 30% by weight of the microsphere. In some embodiments, the cyclodextrin constitutes at least about 5%, at least about 10% of the microsphere, or is about 25% of the microspheres. In some embodiments the microspheres comprise between about 5 and about 50% by weight cyclodextrin, or more specifically between about 5 and 30% by weight cyclodextrin.

The dosage of the therapeutic component in the microsphere is generally in the range from about 0.001 mg to about 400 mg per administered dose, but also can vary from this depending upon the activity of the agent and its solubility. In some embodiments, the dosage of the therapeutic component in the microsphere is 300 mg to about 400 mg per administered dose. In some embodiments, the dosage of the carnosine in the microsphere is 300 mg to about 400 mg per administered dose.

In some embodiments, a double emulsion procedure is used to produce microspheres according to the following general protocol. First, 40 mg carnosine peptide (S212) is dissolved in 0.4 mL of 3% polyvinyl alcohol (PVA) (mw from 10,000-80,000 g/mol) solution in a glass vial. Next, 60 mg poly(lactic-co-glycolic) acid (PLGA) polymer (50:50 Sigma or RG504H) is added with cyclodextrin (CD) in 1 mL dichloromethane (DCM) dropwise while stirring at 1000 RPM, and then stirred for 5 minutes with the cap closed at 1000 RPM. In a separate container, 15 mL of 2% PVA with CD solution is stirred at 500 RPM. The first emulsion is then transferred to the container comprising PVA solution while stirring to form a second emulsion. The second emulsion is then stirred for 72 hours at 500 RPM with the cap open and allowed to evaporate.

### Methods of Treatment

According to some aspects, the present disclosure provides a method of using the therapeutic agent-containing microspheres disclosed herein to treat a neurodegenerative disease or condition. Several neurodegenerative diseases may be treated, ameliorated, or prevented, or the effects minimized, using different embodiments of the microsphere compositions disclosed herein including, for example, Alzheimer's disease and Parkinson's disease. Generally, the treatment may be given in a single dose or multiple administrations, i.e., once, twice, three or more times daily, weekly, or monthly, over a period of time. For chronic disorders such as those diagnosed with, or at risk for, Alzheimer's disease, stroke or Parkinson's disease, the treatment may consist of at least one dose per day over an extended period of time.

The method disclosed herein delivers the therapeutic agent locally or systemically in a sustained manner to a subject, such as a mammal. In some embodiments, the microsphere composition is delivered orally, intramuscularly, intravenously, subcutaneously, intrathecally, and/or by nasal spray. This method allows direct delivery of active ingredients, such as carnosine, systemically or locally.

In some embodiments, liquid dosage forms comprising the microspheres disclosed herein for parenteral administration include, but are not limited to, pharmaceutically acceptable emulsions, microemulsions, solutions, suspensions, syrups, and/or elixirs. In some embodiments, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. In some embodiment, oral compositions can include adjuvants such as wetting agents, emulsifying and suspending agents, sweetening, flavoring, and/or perfuming agents. In certain embodiments for parenteral administration, compositions are mixed with solubilizing agents such as CREMOPHOR®, alcohols, oils, modified oils, glycols, polysorbates, cyclodextrins, polymers, and/or combinations thereof.

In some embodiments, injectable preparations, for example, sterile injectable aqueous or oleaginous suspensions, may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

In some embodiments, injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In some embodiments, liquid dosage forms comprising the microspheres disclosed herein for oral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. In some embodiments, oral compositions can include adjuvants such as wetting agents, emulsifying and suspending agents, sweetening, flavoring, and/or perfuming agents. In certain embodiments for parenteral administration, compositions are mixed with solubilizing agents such as CREMOPHOR®, alcohols, oils, modified oils, glycols, polysorbates, cyclodextrins, polymers, and/or combinations thereof.

In some embodiments, solid dosage forms comprising the microspheres disclosed herein for oral administration include capsules, tablets, pills, powders, and granules. In such solid dosage forms, an active ingredient is mixed with at least one inert, pharmaceutically acceptable excipient such as sodium citrate or dicalcium phosphate and/or fillers or extenders (e.g. starches, lactose, sucrose, glucose, mannitol, and silicic acid), binders (e.g. carboxymethylcellulose, alginates, gelatin, polyvinylpyrrolidinone, sucrose, and acacia), humectants (e.g. glycerol), disintegrating agents (e.g. agar, calcium carbonate, potato or tapioca starch, alginic acid, certain silicates, and sodium carbonate), solution retarding agents (e.g. paraffin), absorption accelerators (e.g. quaternary ammonium compounds), wetting agents (e.g. cetyl alcohol and glycerol monostearate), absorbents (e.g. kaolin and bentonite clay), and lubricants (e.g. talc, calcium stearate, magnesium stearate, solid polyethylene glycols, sodium lauryl sulfate), and mixtures thereof. In the case of capsules, tablets and pills, the dosage form may comprise buffering agents.

In some embodiments, the present disclosure provides an aqueous intranasal formulation comprising the microspheres disclosed herein. In some embodiments, the intranasal formulation comprises a surfactant and/or a thickening agent. In some embodiments, the intranasal formulation may advantageously provide a balance between ease of administration by intranasal delivery and adherence of the formulation to the nasal mucosa. In particular, the intranasal formulation disclosed herein may be administered as a stable intranasal spray yet provide sufficient residence time on the nasal mucosa to allow delivery of the microspheres disclosed herein. Furthermore the intranasal formulations disclosed herein may additionally allow a low or reduced dose of an active agent to be administered, sustained release of the active agent, longer duration of action, and/or a reduced incidence of side effects when compared with other delivery methods.

The thickening agent of the intranasal formulations disclosed herein may modify the viscosity of the formulation to provide improved adherence of the formulation to the nasal mucosa without adversely affecting the ease of administration, in particular administration as an intranasal spray. Without wishing to be bound by theory, the thickening agent may additionally increase the residence time of the formulation on the nasal mucosa, reduce loss of the formulation via mucociliary clearance of the nasal passages and/or improve the trans-nasal absorption. In some embodiments, the thickening agent may comprise about 0.1% to about 10% by weight of the total composition.

In some embodiments, the subject receiving the microspheres disclosed herein receives a therapeutically effective amount of a therapeutic agent for treating the neurological disease/condition for an extended period without requiring additional administrations of the agent or agents. For example, in some embodiments, the patient receives a therapeutically effective amount of a therapeutic agent for at least about one week, at least about one month, at least about two months, at least about 3 months, or at least about 6 months after delivery of the therapeutic agent containing microspheres disclosed herein. Such extended-release times may facilitate successful treatment results, rapid recovery from the condition, and eliminate the need for repeated daily application of the therapeutic agent.

The optimal concentration of the active therapeutic agent will necessarily depend upon the specific neurologic agent used, the characteristics of the patient and the nature of the disease or condition for which the agent is being used. In addition, the concentration will depend upon whether the agent is being employed in a preventive or treatment capacity. Further, the stage of a particular disease or disorder, e.g., early vs. late Alzheimer's disease or Parkinson's disease, may dictate the optimal concentration of the agent.

In some embodiments, the microsphere composition disclosed herein is administered in an amount of 1 mg to 10 mg per kg of subject's body weight.

In some embodiments, the microsphere composition disclosed herein is effective to reduce or eliminate the symptoms of a degenerative neurological disease in a subject in need thereof. In some embodiments, the microsphere composition disclosed herein is effective to reduce or eliminate the symptoms of Parkinson's disease. In some embodiments, the microsphere composition disclosed herein is effective to increase grip strength, improve limb movement, and/or reduce tremor in a subject with Parkinson's disease

In some embodiments, the microsphere formulations disclosed herein may be administered to a subject in need thereof, together with other medication for a discrete period of time, to address specific symptoms. In still other embodiments, the person in need thereof may be treated with both a microsphere disclosed herein and one or more additional medications (administered sequentially or in combination) for the duration of the treatment period. Such combination therapy may be particularly useful, for example, where an additive or synergistic therapeutic effect is desired. In some embodiments, a therapeutic peptide (e.g. carnosine) is administered in combination with L-Dopa for treatment of a neurodegenerative disease or condition. In some embodiments, the therapeutic peptide (e.g. carnosine) is administered in combination with L-Dopa and provides a protective effect against L-Dopa toxicity.

### EXAMPLES

### Example 1—Development of Long-Acting Carnosine Microsphere (S212)

Double Emulsion Microsphere (MS) Procedure: Double emulsion microspheres were produced according to the following general protocol. First, 40 mg carnosine peptide (S212) is dissolved in 0.4 mL of 3% polyvinyl alcohol (PVA) (mw from 10,000-80,000 g/mol) solution in a glass vial. Next, 60 mg poly(lactic-co-glycolic) acid (PLGA) polymer (50:50 Sigma or RG504H) is added (with or without cyclodextrin (CD)) in 1 mL dichloromethane (DCM) dropwise while stirring at 1000 RPM, and then stirred for 5 minutes with the cap closed at 1000 RPM. In a separate container, 15 mL of 2% PVA (with or without out CD) solution is stirred at 500 RPM. The first emulsion is then transferred to the container comprising PVA solution while stirring to form a second emulsion. The second emulsion is then stirred for 72 hours at 500 RPM with the cap open.

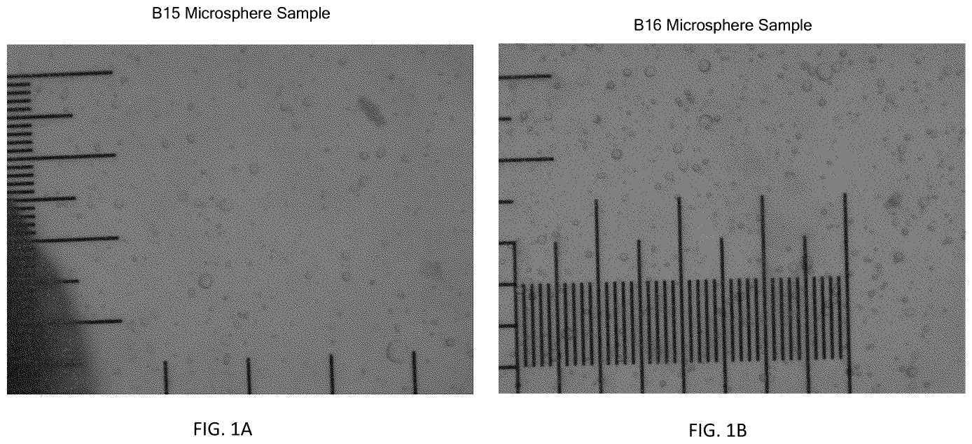

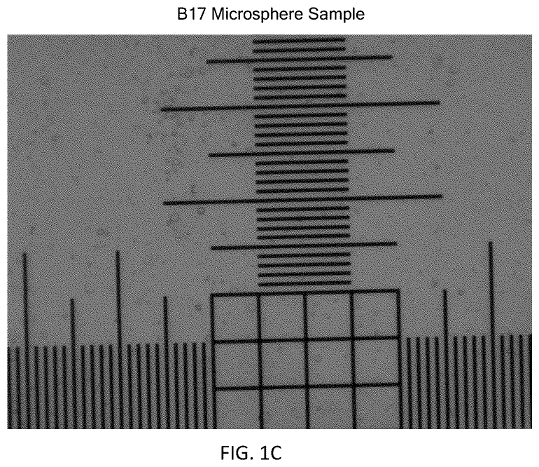

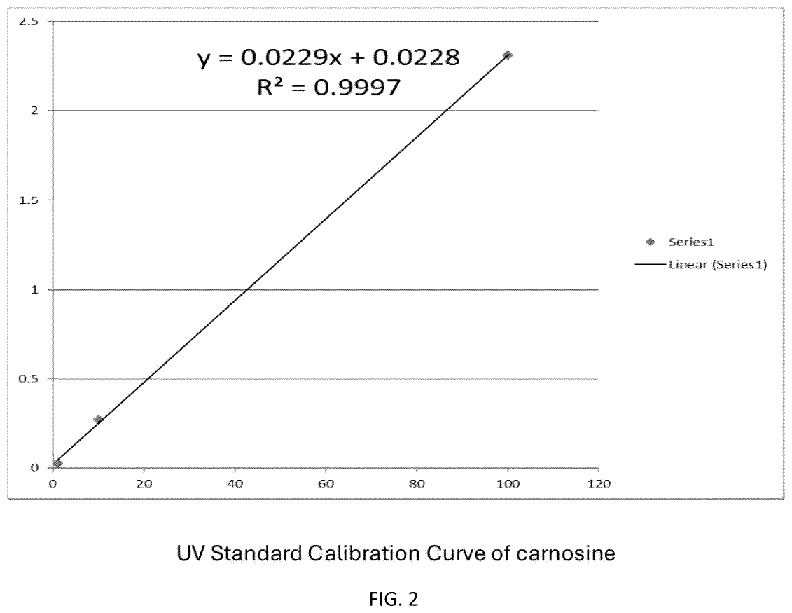

Next, stirring of the second emulsion is stopped and microspheres are allowed to settle to the bottom of the container for about 30 minutes. A sample is taken from the bottom of the container using a pipette. One drop is added to a microscope slide, and observations are recorded from the slide. See FIG. 1A, FIG. 1B, ad FIG. 1C. Then the emulsion is diluted in distilled water in a beaker and microspheres allowed to settle to the bottom (after about 30 minutes). Samples are taken from the top of the mixture of the beaker for absorbance measurements using a UV-Vis spectrophotometer (dilution may be needed if absorbance is too high). To calculate encapsulation efficiency (% encapsulation, % EE), a standard curve is used with equation from the UV standard (y=mx+b), such that y=the observed absorbance, m=slope of the graph, b=y-intercept, and x=the calculated concentration. See Standard Curve in FIG. 2 and Table 1, below. The regression equation was determined to be y=0.0229x+0.001

TABLE 1

| UV Spec 210 nm Standard Solutions | |
|---|---|
| Concentration (μg/mL) | Absorbance |
| 100 | 2.3096 |
| 10 | 0.2727 |
| 1 | 0.0265 |

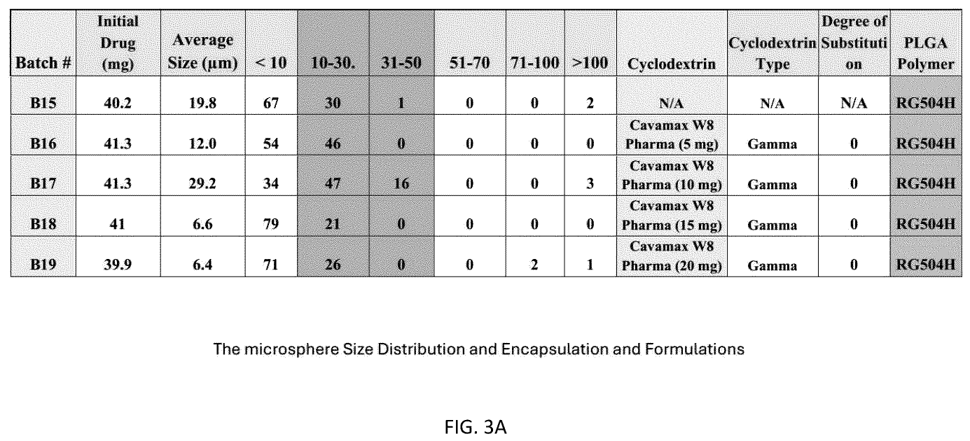

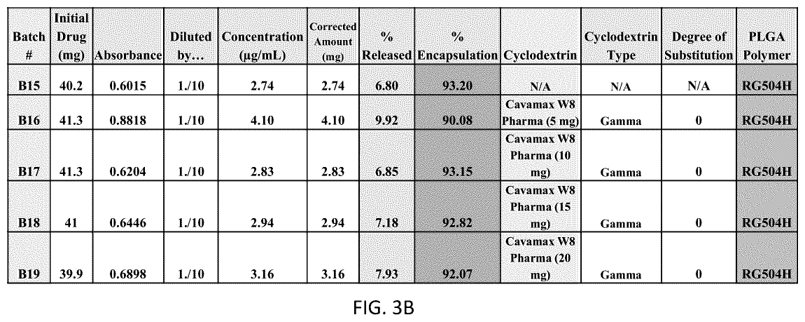

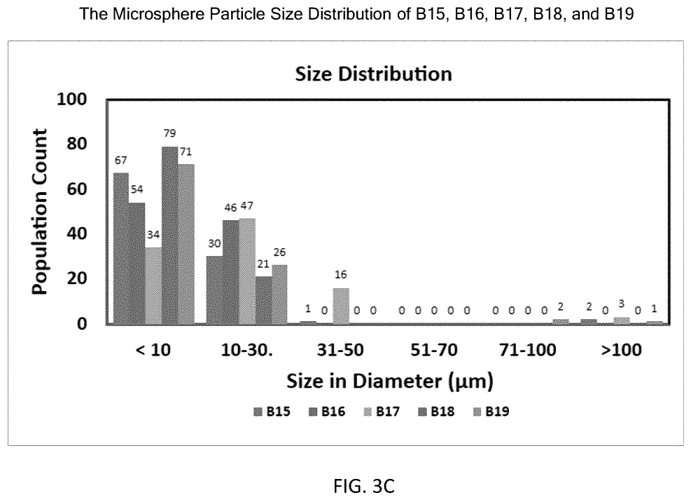

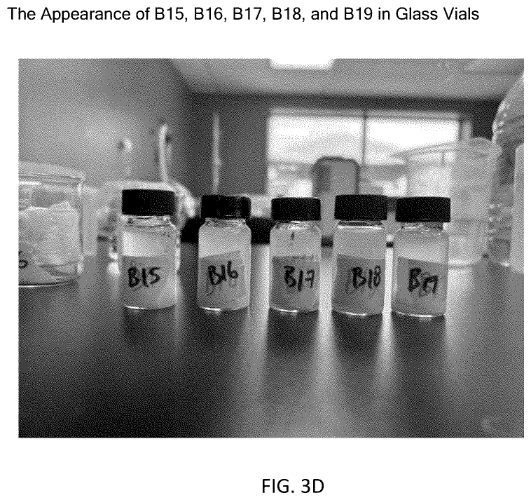

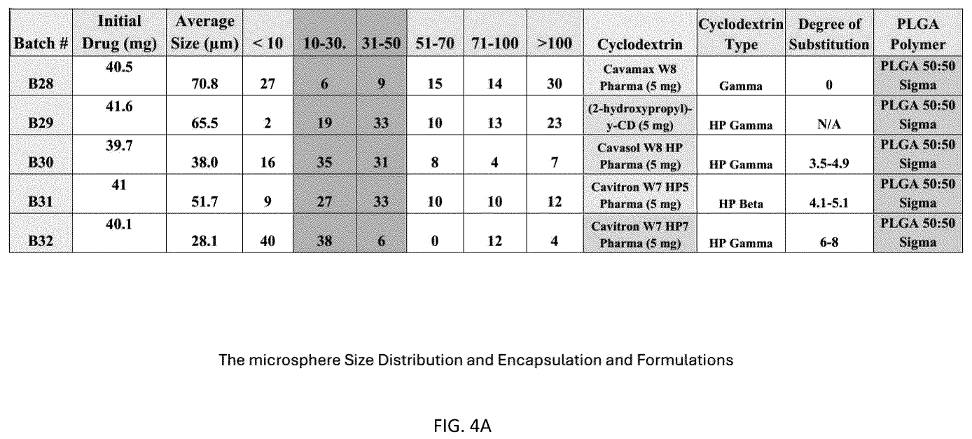

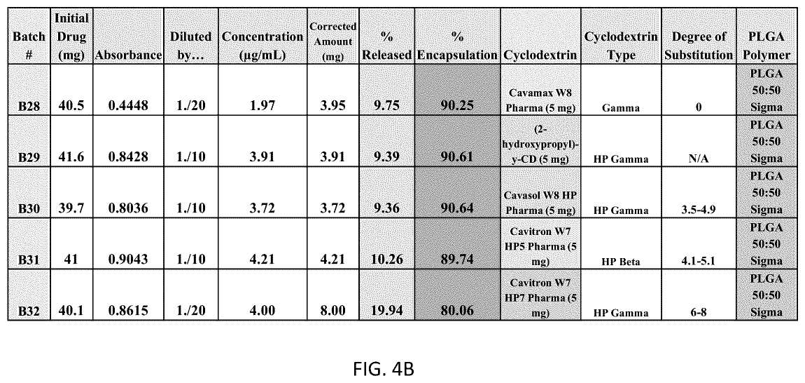

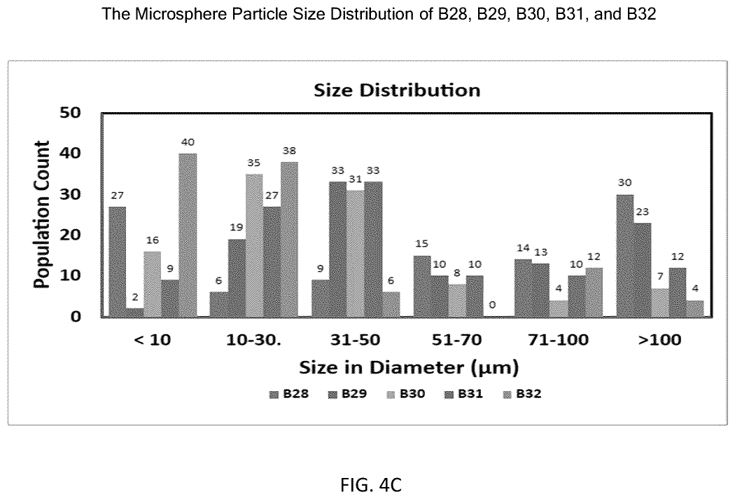

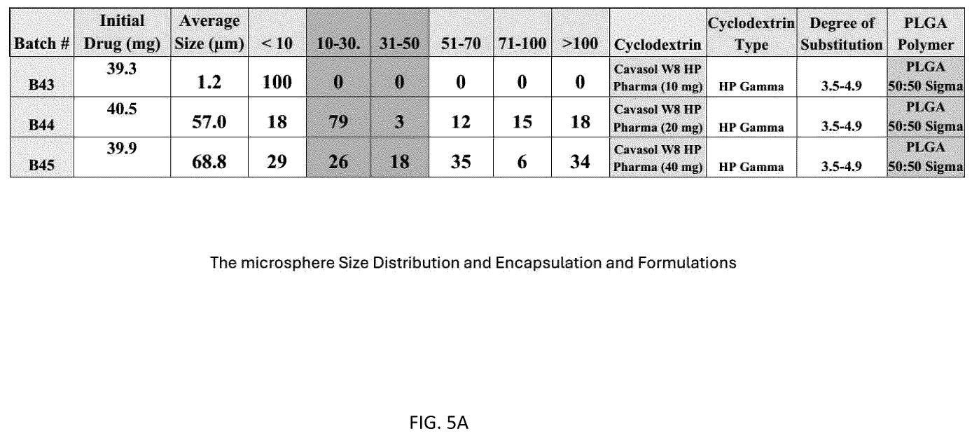

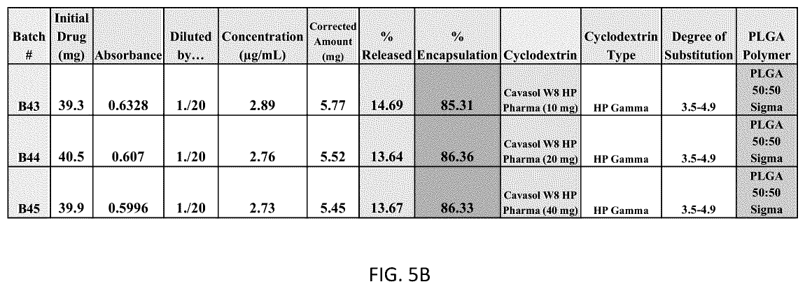

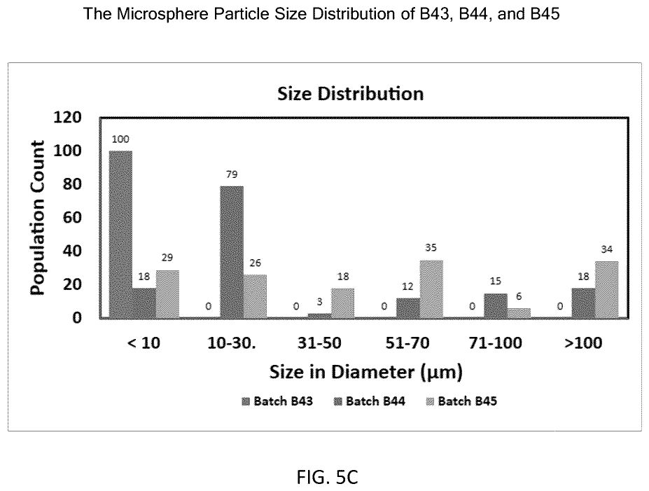

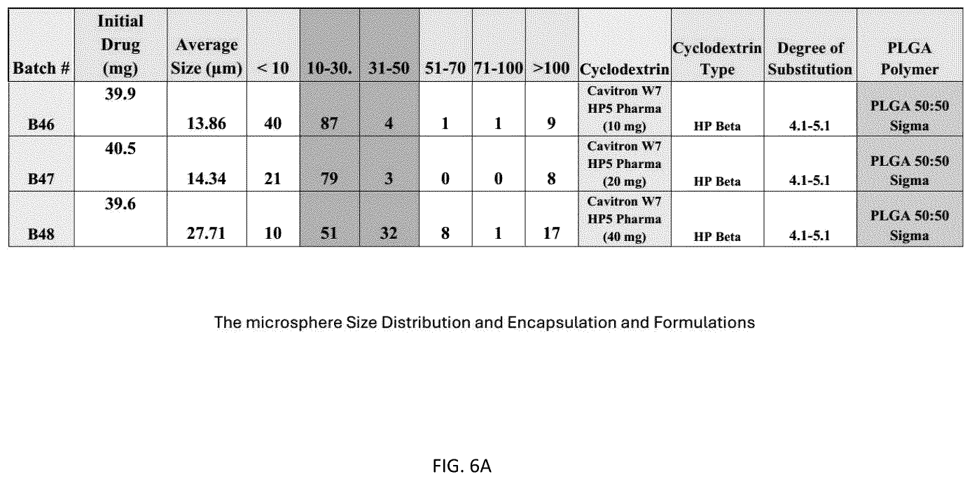

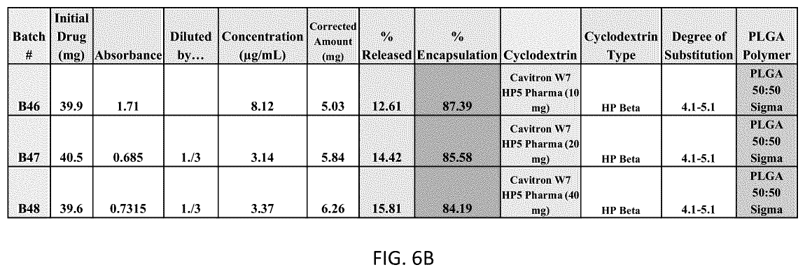

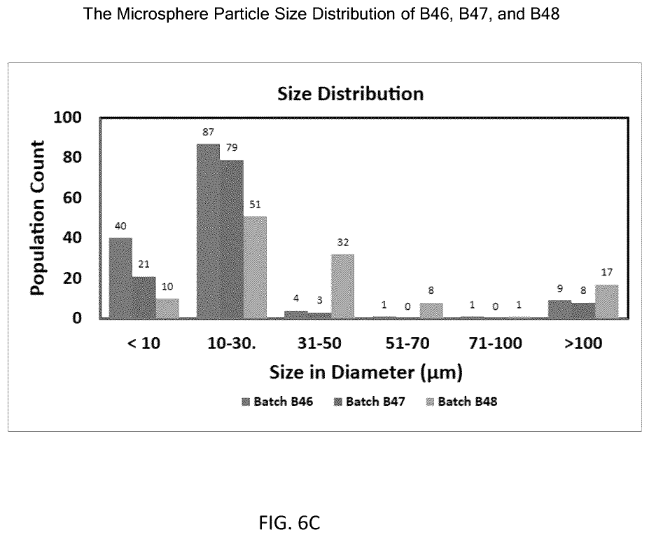

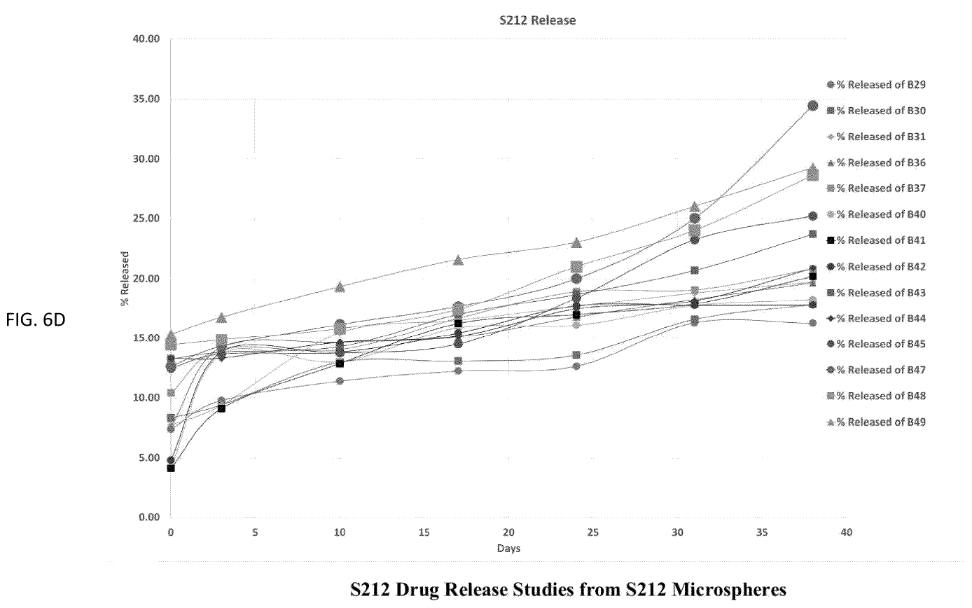

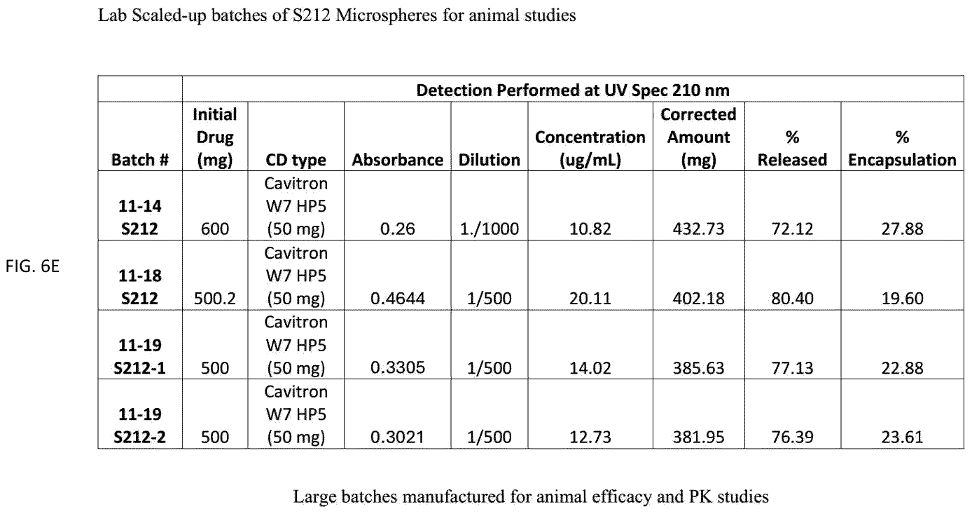

The compositions of various batches (labeled B15, B16, etc.) produced by the general protocol (above) and the resulting characteristics of the microspheres produced is shown FIG. 3 to FIG. 6.

### Lyophilization Procedure

Lyoprotectant Addition: Add a lyoprotectant as disclosed herein at a concentration of 2% (w/v) relative to the mass of the microspheres. For example, if there is 1 g of microspheres, add 20 mg of lyoprotectant to the suspension.

Water Removal and Pre-Freezing: Allow the microspheres to settle for 3-5 hours and remove as much supernatant as possible, leaving approximately 10% of the original water content (10 mL of water for every 100 mL of suspension). Transfer the microsphere suspension to a Falcon tube, allow them to settle again, and decant most of the water while leaving enough (about 10 mL) for freezing.

Freezing: Freeze the microsphere suspension at a temperature of −20° C., ensuring that the microspheres are fully frozen before transferring to the lyophilizer.

Lyophilization: Set the lyophilizer to −40° C. and low atmospheric pressure. Freeze-dry the sample for 1-4 days, depending on the volume of water remaining in the microspheres. The process is complete when a solid, white, powder-like sample is formed, indicating successful removal of moisture. The lyophilized microspheres are transferred to the vial and sealed and stored at −20 degree C.

### Example 2—Neural Cell Gene Expression

### Carnosine Stock:

A stock of Carnosine was made at 884 mM=884 000 uM, it is “10×”, 20% mass/volume in a special media. This medium and all additives are from Stemcell Technologies and is composed of BrainPhys™ Neuronal Medium (Catalog #05795), with NeuroCult™ SM1 Neuronal Supplement (Catalog #05711), N2 Supplement-A (Catalog #07152), Human Recombinant BDNF (Catalog #78005), Human Recombinant GDNF (Catalog #78058), Dibutyryl-CAMP (Catalog #73882), Ascorbic Acid (Catalog #72132).

### Cell Differentiation and Cell Culture:

Astrocytes and Forebrain neurons were differentiated from iPSC-Derived Neural Progenitor Cells (catalog #200-0620) according to vendor protocol (Stemcell Technologies).

Microglia were differentiated from iPSC-Derived Healthy control human iPSC line SCTi003-A (Catalog #200-0510) according to vendor protocol (Stemcell Technologies).

The differentiated Astrocytes/Forebrain neurons/Microglia were co-cultured in BrainPhys™ Neuronal Medium complete medium which consists of BrainPhys™ Neuronal Medium (Catalog #05795), with NeuroCult™ SM1 Neuronal Supplement (Catalog #05711), N2 Supplement-A (Catalog #07152), Human Recombinant BDNF (Catalog #78005), Human Recombinant GDNF (Catalog #78058), Dibutyryl-cAMP (Catalog #73882), Ascorbic Acid (Catalog #72132).

### Experiment:

The experiment contains 4 different cell culture sets: 1. co-culture of Astrocytes/Forebrain neurons/Microglia; 2. Microglia alone; 3. Astrocytes alone; 4. Forebrain neurons alone.

The co-culture of Astrocytes/Forebrain neurons/Microglia as well as the 3 types of cells cultured alone were all grown as: 1) half grown in BrainPhys™ Neuronal Medium complete medium with 1 uM final Amyloid 42-1 Inactive peptide (Catalog #Ab120481, vendor #Abcam) for 24 h, and 2) half in BrainPhys™ Neuronal Medium complete medium with 1 uM final Amyloid Plaque 1-42 Active peptide (Catalog #Ab 120301, vendor #Abcam), with TNF-alpha at 100 ng/ml (Catalog #Ab259410, vendor #Abcam) and with IFN-gamma at 100 ng/ml (catalog #Ab259377, vendor #Abcam) for 24 h.

After the 24 h, the two conditions were each split in 4 different groups: 1. Negative control (0 mM carnosine); 2. Carnosine was added at final concentration of 44.2 mM; 3. Carnosine was added at final concentration of 0.844 mM; 4. Carnosine was added at final concentration of 0.00884 mM.

After 48 h of carnosine dosing, the media were discarded, the cells were lysed using lysis buffer (RLA competed with beta-mercaptoethanol) from a kit of total RNA extraction (SV Total RNA Isolation System) as specified by the vendor (Promega). Each condition, each replicate is extracted as a unique RNA sample. Each condition is done in multi-replicates.

After total RNA extraction, RNA is quantified and checked for quality by measuring the Optical Density of each individual RNA sample.

A reverse transcription is performed on each individual RNA sample at 80 ng of total RNA using the SuperScript™ IV VILO™ Master Mix from vendor ThermoFisher Scientific following manufacturer instructions to produce a cDNA equivalent to each RNA sample in a machine used as a thermocycler.

After reverse transcription, each sample is diluted 1:1 with molecular biology grade water. After dilution of the cDNA samples, a real-time PCR is performed on each cDNA sample (4 ng) set as multiplex, meaning two Taqmans are used simultaneously for each sample using a real-time PCR machine; one Taqman is the Eukaryotic 18S rRNA Endogenous Control the other Taqman is the target gene. The real-time PCR data is run as comparative Ct (delta delta Ct).

Real-time PCR data for each couple Eukaryotic 18S rRNA Endogenous Control the other Taqman is the target gene versus target gene Taqman. All target genes Taqmans are for human genes.

Target genes were selected for genes know to play a role or suggested to play a role in microglia or astrocyte or neurons during the course of Alzheimer's disease. The different couples are as follows:
Eukaryotic 18S rRNA Endogenous Control Taqman (VIC™/MGB probe, primer limited, vendor #ThermoFisher Scientific, catalog #4319413E) and h TGF-beta 1 (vendor #ThermoFisher Scientific, Assay ID #Hs00998133_m1)
Eukaryotic 18S rRNA Endogenous Control Taqman (VIC™/MGB probe, primer limited, vendor #ThermoFisher Scientific, catalog #4319413E) and h MMP-9 (vendor #ThermoFisher Scientific, Assay ID #Hs00957562_m1)
Eukaryotic 18S rRNA Endogenous Control Taqman (VIC™/MGB probe, primer limited, vendor #ThermoFisher Scientific, catalog #4319413E) and h IDE (vendor #ThermoFisher Scientific, Assay ID #Hs00610452_m1)
Eukaryotic 18S rRNA Endogenous Control Taqman (VIC™/MGB probe, primer limited, vendor #ThermoFisher Scientific, catalog #4319413E) and h ECE-1 (vendor #ThermoFisher Scientific, Assay ID #Hs01043735_m1)
Eukaryotic 18S rRNA Endogenous Control Taqman (VIC™/MGB probe, primer limited, vendor #ThermoFisher Scientific, catalog #4319413E) and h GDNF (vendor #ThermoFisher Scientific, Assay ID #Hs01931883_s1)
Eukaryotic 18S rRNA Endogenous Control Taqman (VIC™/MGB probe, primer limited, vendor #ThermoFisher Scientific, catalog #4319413E) and h ET-1 (vendor #ThermoFisher Scientific, Assay ID #Hs00174961_m1)
Eukaryotic 18S rRNA Endogenous Control Taqman (VIC™/MGB probe, primer limited, vendor #ThermoFisher Scientific, catalog #4319413E) and h IL-33 (vendor #ThermoFisher Scientific, Assay ID #Hs04931857_m1)
Eukaryotic 18S rRNA Endogenous Control Taqman (VIC™/MGB probe, primer limited, vendor #ThermoFisher Scientific, catalog #4319413E) and h CXCR4 (vendor #ThermoFisher Scientific, Assay ID #Hs00237052_m1)
Eukaryotic 18S rRNA Endogenous Control Taqman (VIC™/MGB probe, primer limited, vendor #ThermoFisher Scientific, catalog #4319413E) and h SDF-1 (vendor #ThermoFisher Scientific, Assay ID #Hs03676656_mH)
Eukaryotic 18S rRNA Endogenous Control Taqman (VIC™/MGB probe, primer limited, vendor #ThermoFisher Scientific, catalog #4319413E) and h SIRT-1 (vendor #ThermoFisher Scientific, Assay ID #Hs01009006_m1)
Eukaryotic 18S rRNA Endogenous Control Taqman (VIC™/MGB probe, primer limited, vendor #ThermoFisher Scientific, catalog #4319413E) and h IL-6 (vendor #ThermoFisher Scientific, Assay ID #Hs00174131_m1)
Eukaryotic 18S rRNA Endogenous Control Taqman (VIC™/MGB probe, primer limited, vendor #ThermoFisher Scientific, catalog #4319413E) and h CXCL10 (vendor #ThermoFisher Scientific, Assay ID #Hs00171042_m1)
Eukaryotic 18S rRNA Endogenous Control Taqman (VIC™/MGB probe, primer limited, vendor #ThermoFisher Scientific, catalog #4319413E) and h IL-10 (vendor #ThermoFisher Scientific, Assay ID #Hs00961622_m1)

t TEST (sample compared to control) was performed for the obtained data, with p value equal of less than 0.05 being considered as specific.

MMP-9, IDE, ECE-1 are proteases described as cleaving Amyloid Plaque.

BDNF is a neuronal growth factor.

ET-1 is a growth factor for Oligodendrocytes. Oligodendrocytes feed and protect neurons.

IL-33 is a described as decreasing Alzheimer's disease-like pathology, including decreasing cognitive decline.

SDF-1 (CXCL12) is the ligand of CXCR4.

SIRT-1 is a survival factor.

IL-6, CXCL10 are cytokines that increase inflammation.

IL-10 is an interleukin that reduces inflammation.

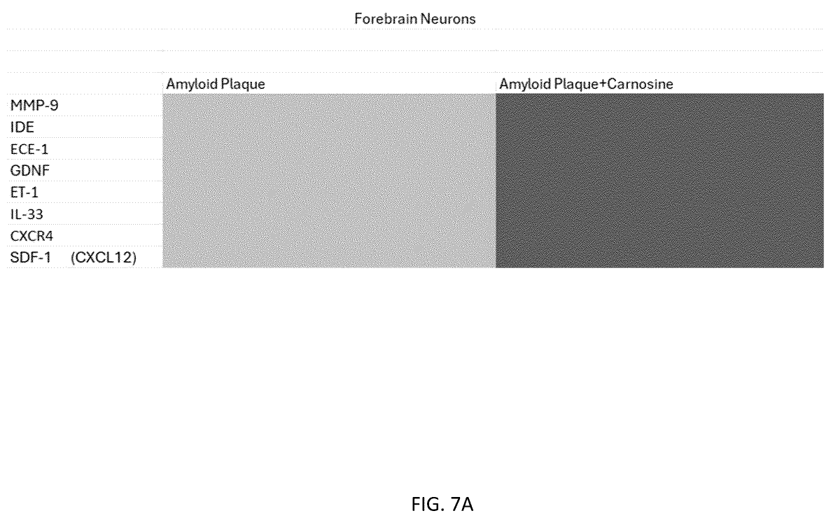

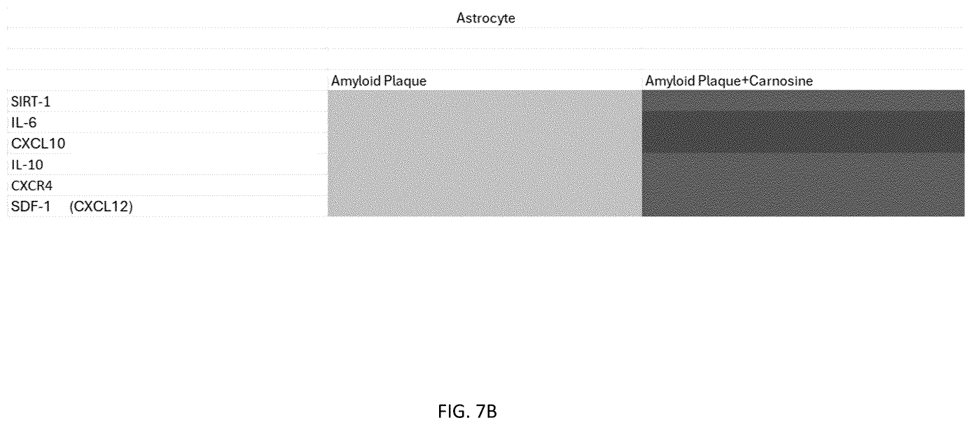

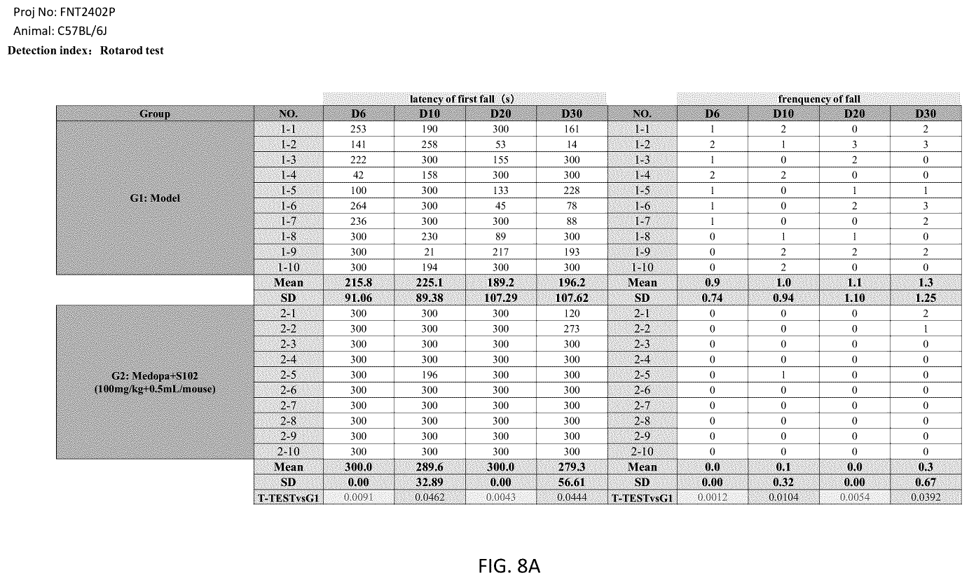

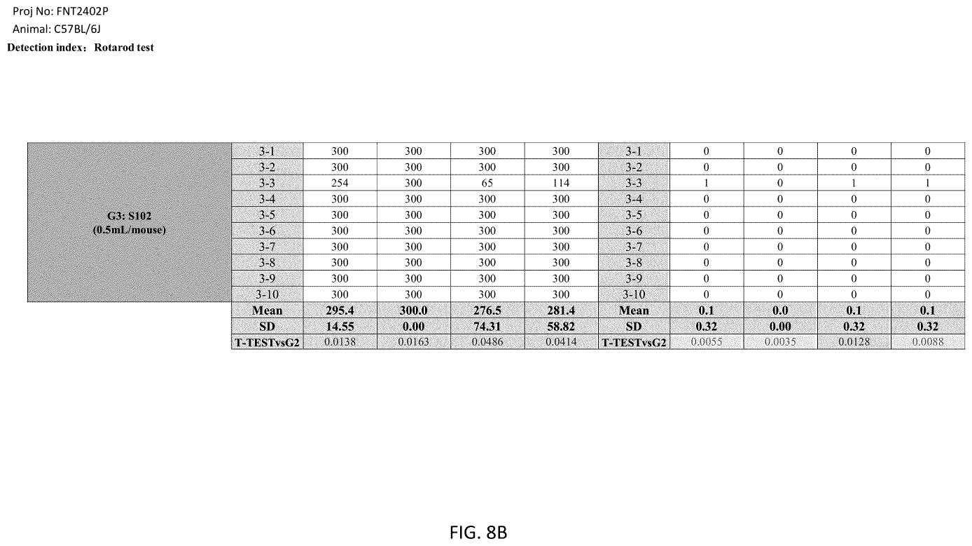

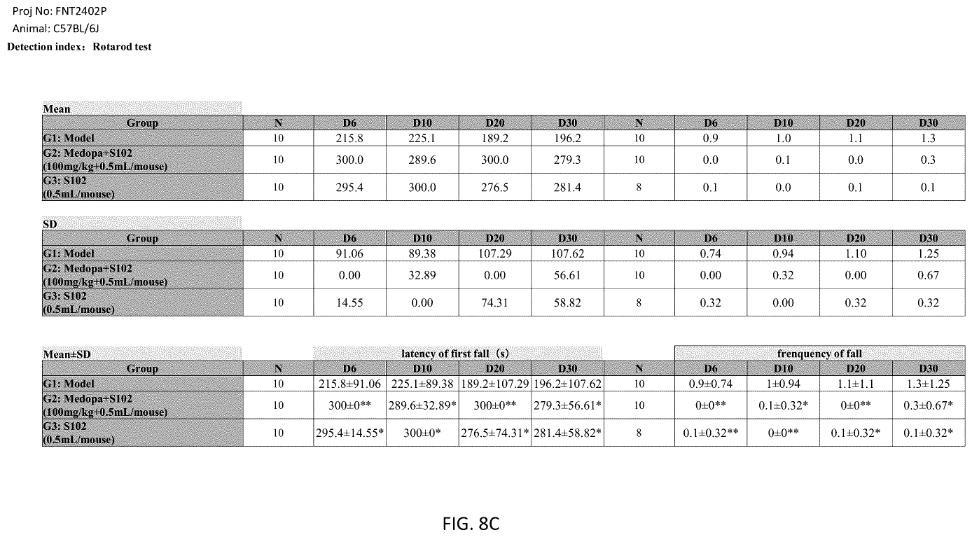

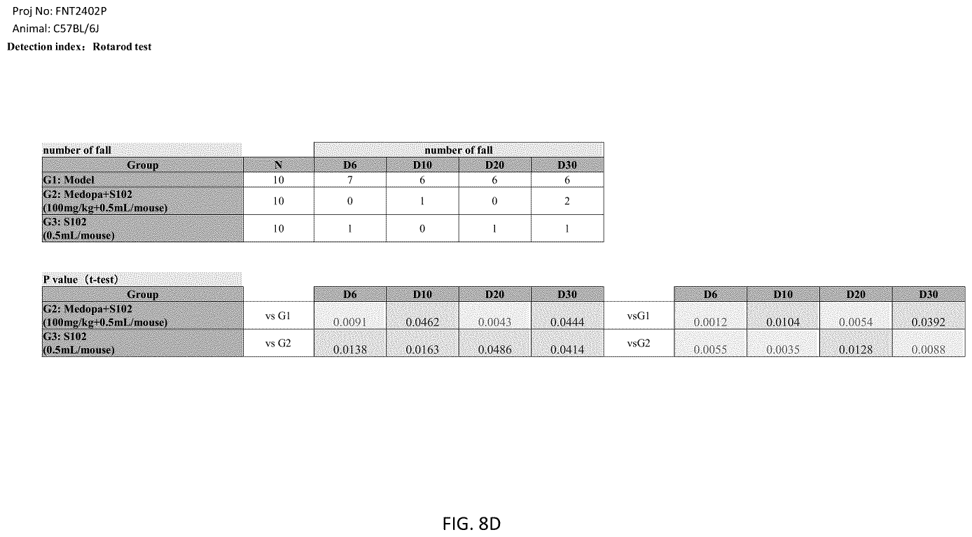

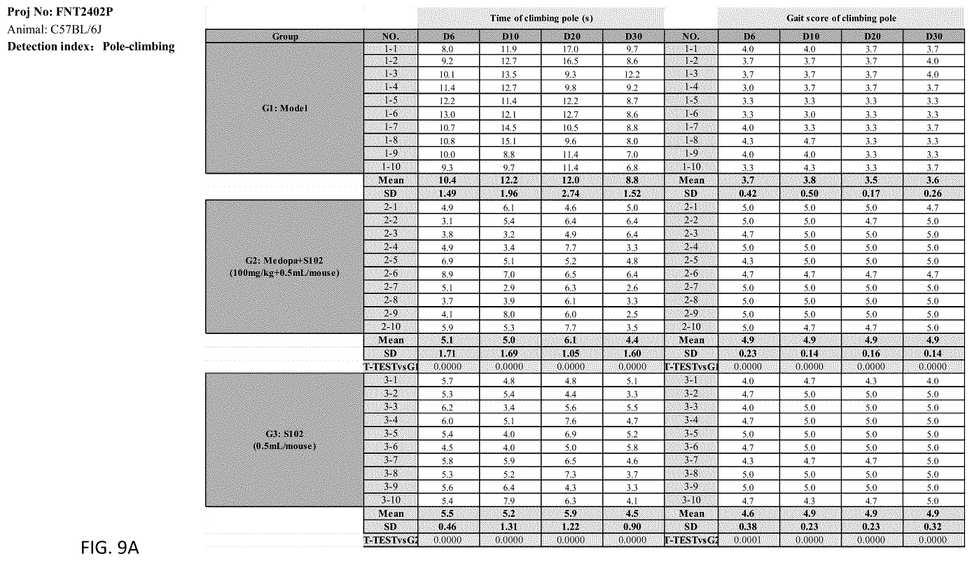

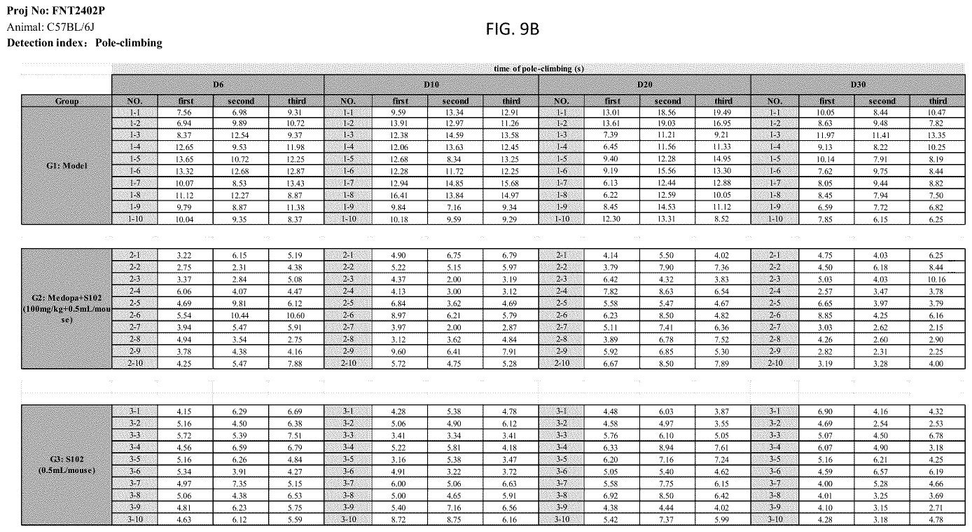

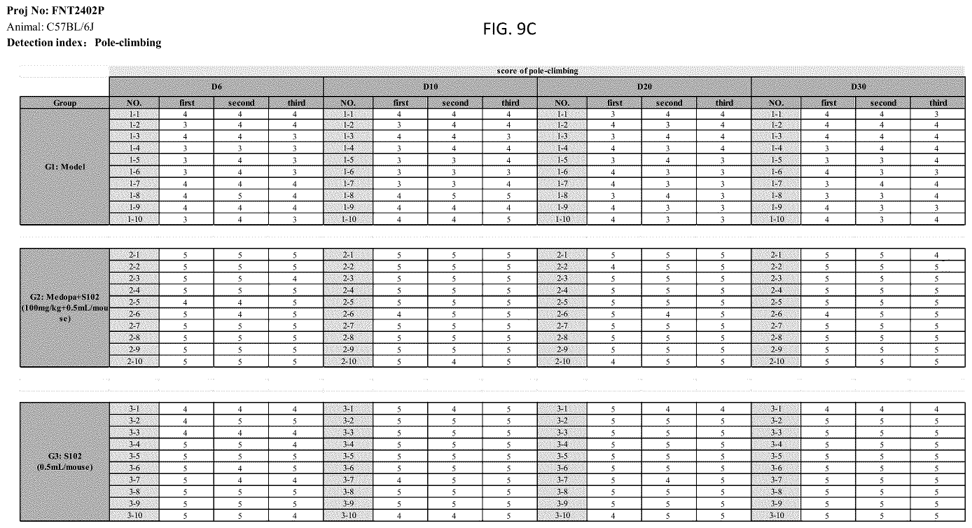

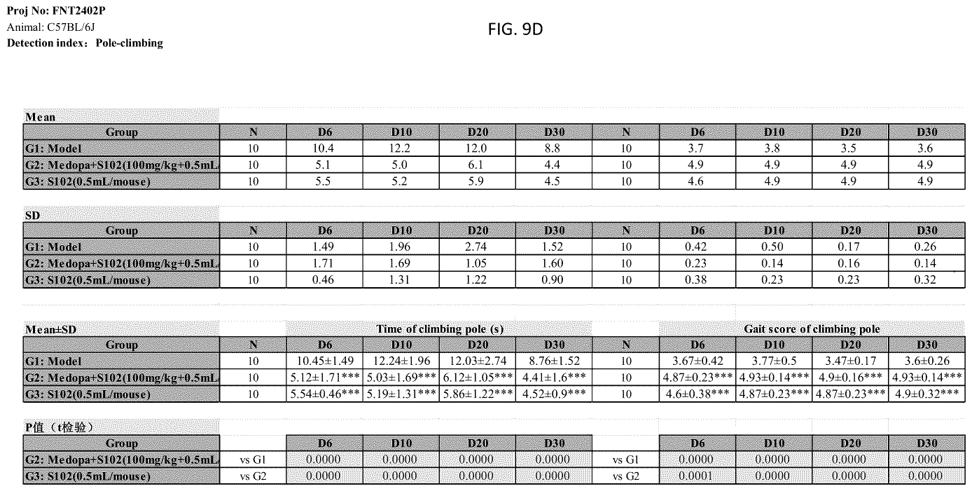

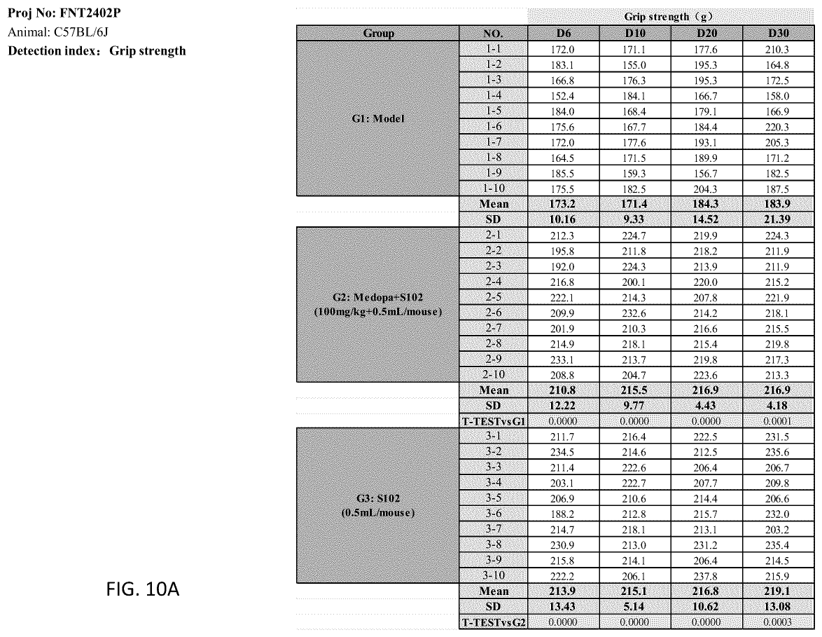

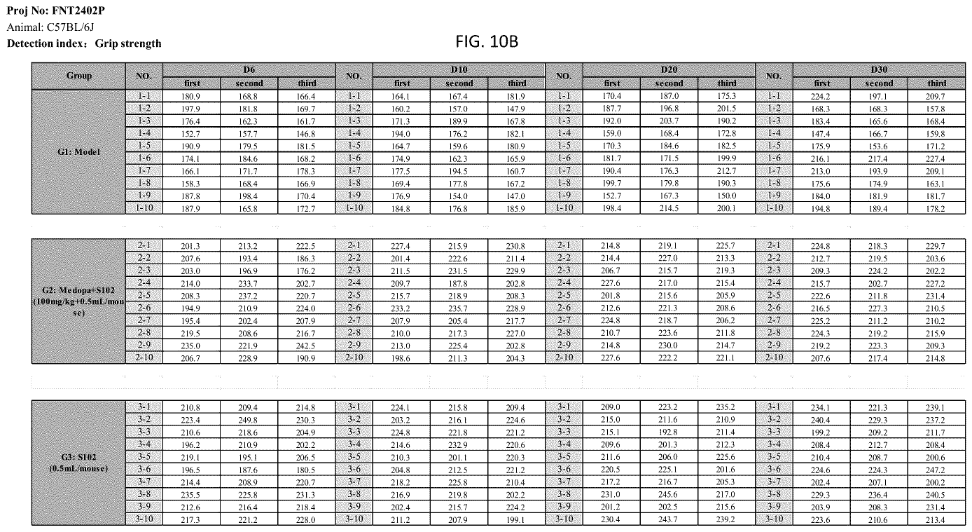

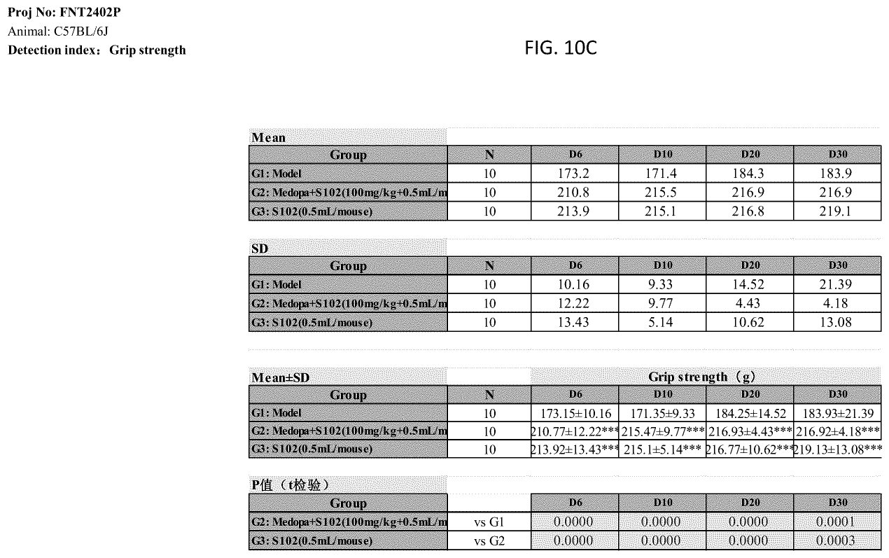

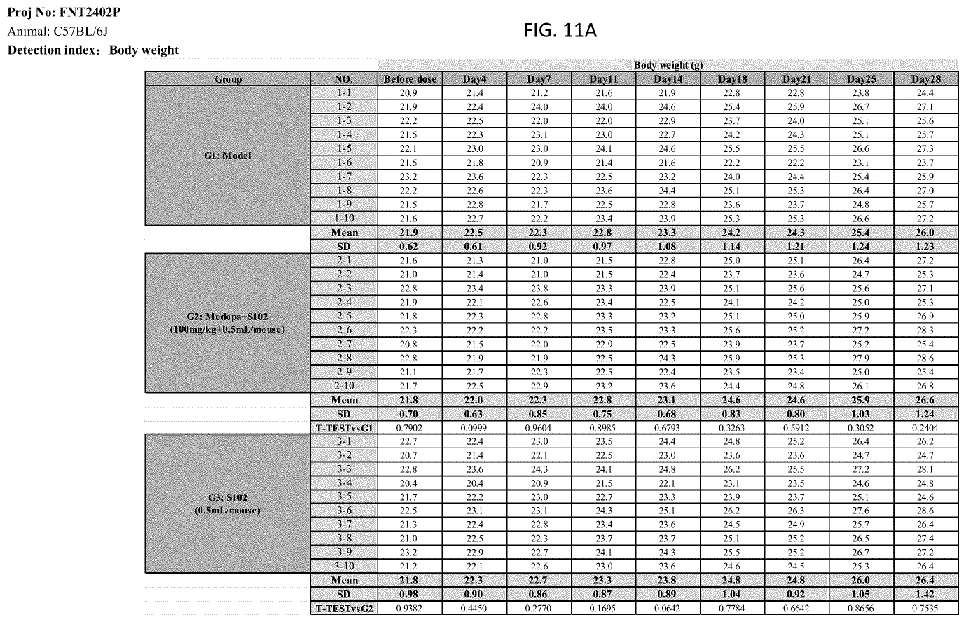

Changes in gene expression is show in FIGS. 7A and 7B. Green represents upregulation; Red represents downregulation.

### Example 3—Pharmacodynamic Study of the Test Article in Parkinson's Disease Model Induced by MPTP in Mice

### Abstract

Objective: To evaluate whether a single subcutaneous injection of the test article has a long-term protective effect on MPTP-induced Parkinson's mice.

Methods: The qualified mice after adaptive training and pre-screening were randomly divided into three groups according to body weight, with 10 mice in each group, which were: (I) model group, (II) S212 microsphere+medopa group (0.5 mL/mouse+100 mg/kg), (III) S212 microsphere group (0.5 mL/mouse). Then the administration and modeling begin, and the modeling was taken after treatment. Mice in the three groups were intraperitoneally injected with MPTP (25 mg/kg, 5 mL/kg) once a day for 10 consecutive days. The frequency of administration of the medopa drug was once a day, and the S212 microsphere were administered on the first day (D1), sixth day (D6) and eighteenth day (D18) of modeling administration. The first day of administration was recorded as D1, and the measurement of the pole test, rotating rod test and grip strength test were taken on the sixth day (D6), tenth day (D10), the twentieth day (D20) and the thirtieth day (D30). The mice's pole-climbing time and pole-climbing gait scores, as well as grip strength values, were recorded. The rotarod test was set at 30 rpm for 5 min, and the time of each mouse falling from the baton, as well as the total number of mice in each group falling from the baton, were observed. After the end of the experimental period, the mice were injected with 4% paraformaldehyde, and the whole brain tissues were extracted and fixed in paraformaldehyde solution. After paraffin embedding and fixation, TH (tyrosine hydroxylase) immunohistochemical staining of the substantia nigra and macula densa in the brain were performed.

Results: (1) the rotarod test: \textcircled{1} at Day 6, Day 10, Day 20 and Day 30, the latency of the first fall in PD mice in both the S212 microsphere+medopa (100 mg/kg+0.5 mL/mouse) group and S212 microsphere (0.5 mL/mouse) group was significantly longer compared to that of the model group (P<0.01, P<0.05 vs Model group). \textcircled{2} at Day 6, Day 10, Day 20 and Day 30, the fall frequency within 5 min in PD mice in both the S212 microsphere+medopa (100 mg/kg+0.5 mL/mouse) group and S212 microsphere (0.5 mL/mouse) group were significantly decreased compared to that of the model group (P<0.01, P<0.05 vs Model group). \textcircled{3} at Day 6, Day 10, Day 20 and Day 30, the fall number within 5 min of PD mice in both the S212 microsphere+medopa (100 mg/kg+0.5 mL/mouse) group and S212 microsphere (0.5 mL/mouse) group were decreased compared to that of the model group. (2) the pole climbing test: \textcircled{1} after three administrations (on Day 1, Day 6 and Day 18), compared with the model group, the climbing time of PD mice in the S212 microsphere+medopa (100 mg/kg+0.5 mL/mouse) group and S212 microsphere (0.5 mL/mouse) group decreased significantly, suggesting that the motor function damage of PD mice had certain improved after treatment. \textcircled{2} after three administrations (on Day 1, Day 6 and Day 18), S212 microsphere+medopa (100 mg/kg+0.5 mL/mouse) group and S212 microsphere (0.5 mL/mouse) group showed a significant increase in gait score of PD mice compared with the model group. (3) the grip strength test: compared with the model group, both the S212 microsphere+medopa (100 mg/kg+0.5 mL/mouse) and S212 microsphere (0.5 mL/mouse) significantly improved the grip strength of PD mice (P<0.001 vs Model group). (4) the body weight: during the administration period, the body weight of PD mice rised slowly. Compared with the model group, the S212 microsphere+medopa (100 mg/kg+0.5 mL/mouse) and S212 microsphere (0.5 mL/mouse) had no significant effect on the body weight of PD mice.

Conclusion: In this study, S212 microsphere+medopa (100 mg/kg+0.5 mL/mouse) and S212 microsphere (0.5 mL/mouse) via subcutaneous injection for 3 times (on Day 1, Day 6, Day18) could improve the limb movement injury of PD model mice induced by MPTP.

### Objective

To evaluate whether a single subcutaneous injection of the test substance has a long-term protective effect on MPTP-induced Parkinson's mice.

### Compound and Preparation

Test materials (S212 microspheres) was as disclosed in Example 1.

Positive drug: Name: Dobasic Hydrazide Tablets (Medopa®) Manufacturer: Shanghai Roche Pharmaceutical Co. Description: Reddish tablets with coloring agent. Batch number: YT1363. Specification: 250 mg/tablet. Storage conditions: Shield from light, seal and store in a cool dry place.

Molding agent: Name: 1-methyl-4-phenyl-1,2,3,6-tetrahydropyridine hydrochloride (MPTP hydrochloride); Manufacturer: Sigma-Aldrich; Description: white or off-white powder; Molecular Formula: C12H15N—HCl; Molecular weight: 209.72; Specification: 1000 mg/vial; CAS-No: 23007-85-4; Storage condition: Protected from light, room temperature.

### Main Equipment and Agents

Equipment: A. Name: Rotating rod fatigue meter; Manufacturer: Huabei Zhenghua Biological Instrument Co.; Type: YLS-4C; B. Name: Mouse Grip Tester; Manufacturer: Jinan Yiyan Technology Development Co.; Type: YLS-13A; C. Name: Mouse pole climbing instrument; Specification: Wooden pole (1 cm in diameter, 50 cm in height) with small ball at the top; Source: Self-made.

### Agents

| Name | Manufacturer | Batch number |
|---|---|---|
| CMC-Na | Shanghai Aladdin | E2411161 |
| | Biochemical Technology Co., LTD | |
| Sodium chloride | Zhejiang Tianrui | 124071206 |
| injection | Pharmaceutical Co., LTD | |
| Sterilized Water | Anhui Shuang He | 24091402A |
| for Injection | Pharmaceutical Co., LTD | |

### Animals and Husbandry

Animals: Strain and Species: C57BL/6J mice; Grade: SPF; Gender: Male; Source: Zhejiang Vital River Laboratory Animal Technology Co., Ltd.; Laboratory Animal Quality Certification No: 20241128Abzz0619000735; Number of animals: 40 C57BL/6J mice were ordered; Age at animals arrive: 7˜8 w; Upon arrival at the animal facility, animals were housed 5 mouse per; cage and acclimated for three to seven days.

Environment: Environmental controls for the animal room were set to maintain a temperature of 20˜25° C., humidity of 40˜70%, and a 12-hour light/12-hour dark cycle. The 12-hour dark cycle may be temporarily interrupted to accommodate study procedures.

Food and water: SPF mice growth breeding feed (Jiangsu Xietong Pharmaceutical Bio-engineering Co., Ltd.) was provided ad libitum throughout the in-life part of the study. Reverse Osmosis water was available ad libitum.

### Study Design

Rotarod test: On the first day, mice were placed on the rotating bar of the instrument and the instrument were turned on. Rotation speed: 14 rpm, timed for 6 min. each mouse was trained once; on the second day, mice were placed in the same way, and the rotation speed was increased to 18 rpm, timed for 4 min; on the third day, mice were continued to be placed in the same way, and the rotation speed was increased to 30 rpm, timed for 5 min, and the mice with fallen rods were rejected.

Adaptive training method of pole climbing instrument: The qualified mice selected in the rotating rod training were subjected to adaptive training with the pole climbing instrument for 3 consecutive days to ensure that all mice knew how to climb down the pole. Mice were placed head-down on a small ball at the top of a wooden pole (1 cm in diameter and 50 cm in height) to climb down naturally along the pole, and the movement behavior of mice were observed during the climbing process. Train three times a day, three days in a row. Pole climbing gait was scored according to the following criteria: 5 points: Use all four limbs and crawl down step by step in coordination. 4 points: crawling down step by step, but with sliding hind legs. 3 points: Climb half the distance after sliding down, but can hold the pole. 2 points: Sliding behavior occurs after climbing half the distance. 1 point: Can not grasp the pole after climbing half the distance, fall from the pole. Pole climbing test screening: After the third day of pole climbing training, the unqualified animals that could not climb down smoothly or stayed on the pole for too long were eliminated.

### Animal Groups and Drugs

Groups were formed according to the following table:

| | | | | | | Dosing route, |
|---|---|---|---|---|---|---|
| | | Animal | | | Concentration | frequency& |
| Group | Treatment | Number | Dose | Dose Volume | (mg/mL) | period |
| 1 | saline | 10 | — | 0.5 mL/mouse | — | sc, day 1, day 6, |
| | | | | | | day 18 |
| 2 | S212 | 10 | 0.5 mL/ | 0.5 mL/mouse + | n/a + | sc, day 1, day 6, |
| | microsphere + | | mouse + | 10 mL/kg | 10 mg/mL | day 18 |
| | medopa | | 100 | | | (S212) + |
| | | | mg/kg | | | i.g, qd*30 days |
| | | | | | | (medopa) |
| 3 | S212 | 10 | 0.5 mL/ | 0.5 mL/mouse | n/a | sc, day 1, day 6, |
| | microsphere | | mouse | | | day 18 |

### Drug Configuration

Test article preparation: microsphere formulations were as disclosed in Example 1.

Preparation of positive drugs: 1 tablet of medopa was ground in a mortar, mixed thoroughly and evenly, and added 25 ml of 0.5% CMC-Na, mixed by ultrasound, prepared before use.

Preparation of MPTP: Weigh 25 mg MPTP hydrochloride, dissolve with sodium chloride injection, constant volume to 5 mL, prepare before use, store away from light.

Treatments: The qualified mice after adaptive training and pre-screening were randomly divided into three groups according to body weight, with 10 mice in each group, which were: (I) model group, (II) S212 microsphere+medopa group, (III) S212 microsphere group. Then the administration and modeling begin, and the modeling was taken after treatment. Mice in the three groups were intraperitoneally injected with MPTP (25 mg/kg, 5 mL/kg) once a day for 10 consecutive days, the frequency and the period of treatment were as described above. The first day of administration was recorded as D1, and the measurement of the pole test, rotating rod test and grip strength test were taken on the sixth day (D6), tenth day (D10), the twentieth day (D20) and the thirtieth day (D30). The mice's pole-climbing time and pole-climbing gait scores, as well as grip strength values, were recorded. The rotarod test were set at 30 rpm for 5 min, and the time of each mouse falling from the baton, as well as the total number of mice in each group falling from the baton, were observed. After the end of the experimental period, the mice were injected with 4% paraformaldehyde, and the whole brain tissues were extracted and fixed in paraformaldehyde solution. After paraffin embedding and fixation, TH (tyrosine hydroxylase) immunohistochemical staining of the substantia nigra and macula densa in the brain were performed.

Statistical analysis: The results were expressed as Mean±SD. Statistical significance was considered when the P value was less than 0.05. The final data was plotted with GraphPad Prism software.

### Results

### The Effect of S212 Microsphere Via Subcutaneous Injection for 3 Times (on Day 1, Day 6 and Day 18) on the Rotarod Test in MPTP-Induced PD Model Mice

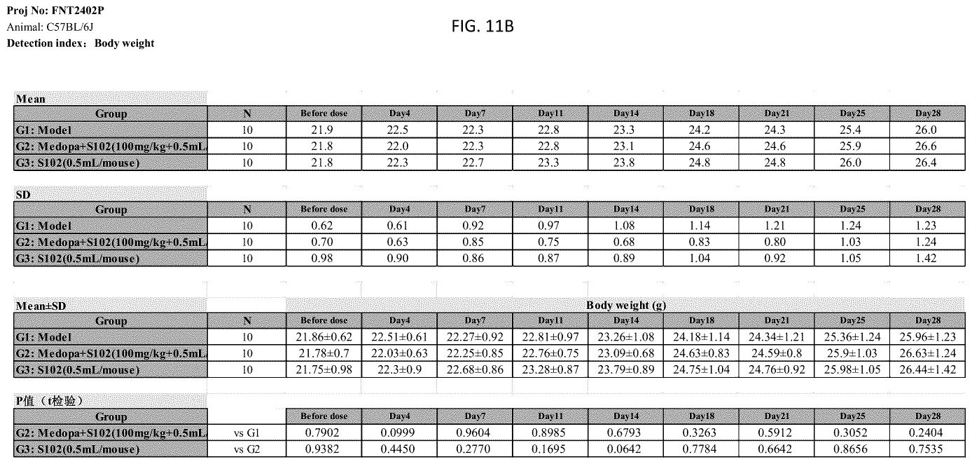

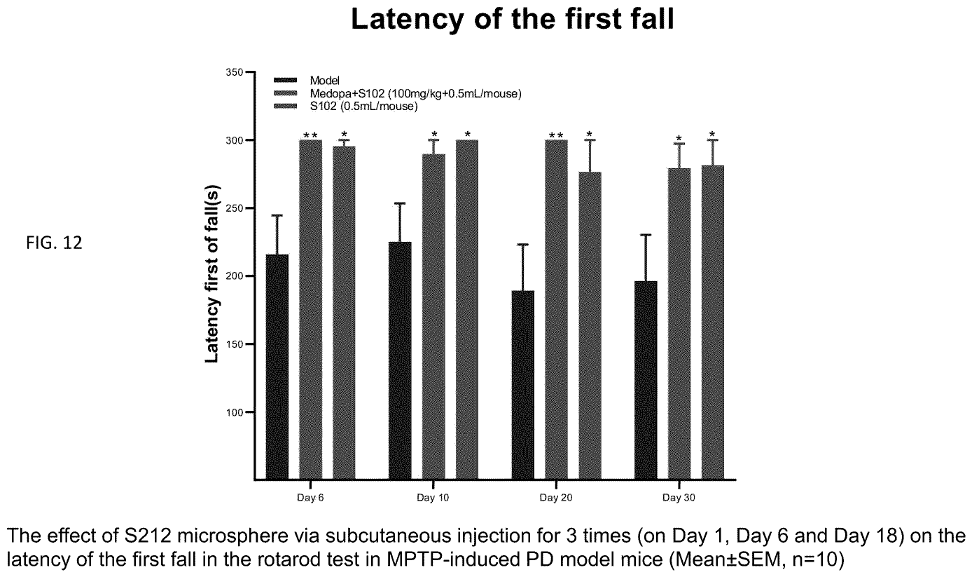

There were three main index of the rotarod test: \textcircled{1} the first time of the mouse falled from the rod (the latency of the first fall, seconds), \textcircled{2} frequency of the fall each mouse falled from the rotarod fall in 5 min (the fall frequency), and \textcircled{3} the fall number within 5 min of PD mice. These three indicators should be considered together when evaluating drug efficacy. \textcircled{1} The effect on the latency of the first fall: according to the data in FIG. 12 and Tab. 1-1, at Day 6, Day 10, Day 20 and Day 30, the latency of the first fall in PD mice in both the S212 microsphere+medopa (100 mg/kg+0.5 mL/mouse) group and S212 microsphere (0.5 mL/mouse) group was significantly longer compared to that of the model group (P<0.01, P<0.05 vs Model group). \textcircled{2} The effect on the fall frequency within 5 min: according to the data in Tab. 1-2, at Day 6, Day 10, Day 20 and Day 30, the fall frequency within 5 min in PD mice in both the S212 microsphere+medopa (100 mg/kg+0.5 mL/mouse) group and S212 microsphere (0.5 mL/mouse) group were significantly decreased compared to that of the model group (P<0.01, P<0.05 vs Model group). \textcircled{3} The effect on the fall number within 5 min: according to the data in Tab. 1-3, at Day 6, Day 10, Day 20 and Day 30, the fall number within 5 min of PD mice in both the S212 microsphere+medopa (100 mg/kg+0.5 mL/mouse) group and S212 microsphere (0.5 mL/mouse) group were decreased compared to that of the model group. See FIG. 12.

TABLE 1-1
The effect of S212 microsphere via subcutaneous injection for 3 times
(on Day 1, Day 6 and Day 18) on the latency of the first fall in the
rotarod test in MPTP-induced PD model mice (Mean ± SD, n = 10)

| | Latency of the first fall (s) | | | |
|---|---|---|---|---|
| Group | Day 6 | Day 10 | Day 20 | Day 30 |
| G1: Model | 215.8 ± 91.06 | 225.1 ± 89.38 | 189.2 ± 107.29 | 196.2 ± 107.62 |
| G2: Medopa + S102 | 300 ± 0** | 289.6 ± 32.89* | 300 ± 0** | 279.3 ± 56.61* |
| (100 mg/kg + 0.5 mL/mouse) | | | | |
| G3: S102 (0.5 mL/mouse) | 295.4 ± 14.55* | 300 ± 0* | 276.5 ± 74.31* | 281.4 ± 58.82* |

**P < 0.001,
*P < 0.05 vs. Model

TABLE 1-2
The effect of S212 microsphere via subcutaneous injection for 3 times
(on Day 1, Day 6 and Day 18) on the frequency of the fall in the
rotarod test in MPTP-induced PD model mice (Mean ± SD, n = 10)

| | Frequency of the fall | | | |
|---|---|---|---|---|
| Group | Day 6 | Day 10 | Day 20 | Day 30 |
| G1: Model | 0.9 ± 0.74 | 1 ± 0.94 | 1.1 ± 1.1 | 1.3 ± 1.25 |
| G2: Medopa + S102 | 0 ± 0** | 0.1 ± 0.32* | 0 ± 0** | 0.3 ± 0.67* |
| (100 mg/kg + 0.5 mL/mouse) | | | | |
| G3: S102 (0.5 mL/mouse) | 0.1 ± 0.32** | 0 ± 0** | 0.1 ± 0.32* | 0.1 ± 0.32* |

**P < 0.001
*P < 0.05 vs. Model

TABLE 1-3
The effect of S212 microsphere via subcutaneous injection for 3 times
(on Day 1, Day 6 and Day 18) on the Number of the fall in the rotarod
test in MPTP-induced PD model mice (Mean ± SD, n = 10)

| | Number of falls | | | |
|---|---|---|---|---|
| Group | Day 6 | Day 10 | Day 20 | Day 30 |
| G1: Model | 7 | 6 | 6 | 6 |
| G2: Medopa + S102 | 0 | 1 | 0 | 2 |
| (100 mg/kg + 0.5 mL/mouse) | | | | |
| G3: S102 (0.5 mL/mouse) | 1 | 0 | 1 | 1 |

In summary, in the rotarod test, S212 microsphere+medopa (100 mg/kg+0.5 mL/mouse). S212 microsphere (0.5 mL/mouse) via subcutaneous injection for 3 times (on Day 1, Day 6 and Day18) of administration can improve the limb movement injury induced by MPTP in PD model mice, showed as prolonging the latency of the first fall, reducing the fall frequency and decreasing the fall number of PD mice.

### The Effect of S212 Microsphere Via Subcutaneous Injection for 3 Times (on Day 1, Day 6 and Day 18) on the Pole Climbing Test in MPTP-Induced PD Model Mice

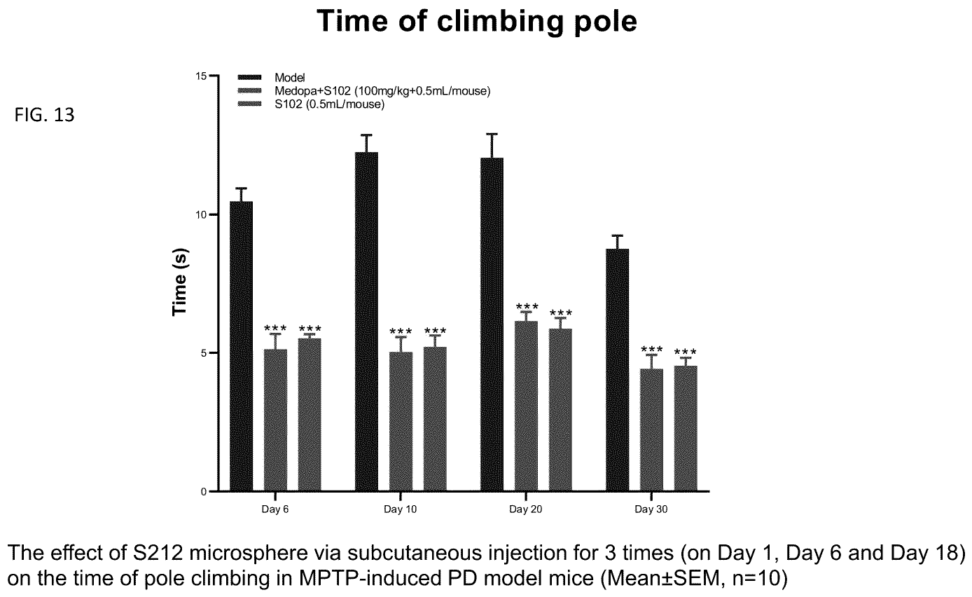

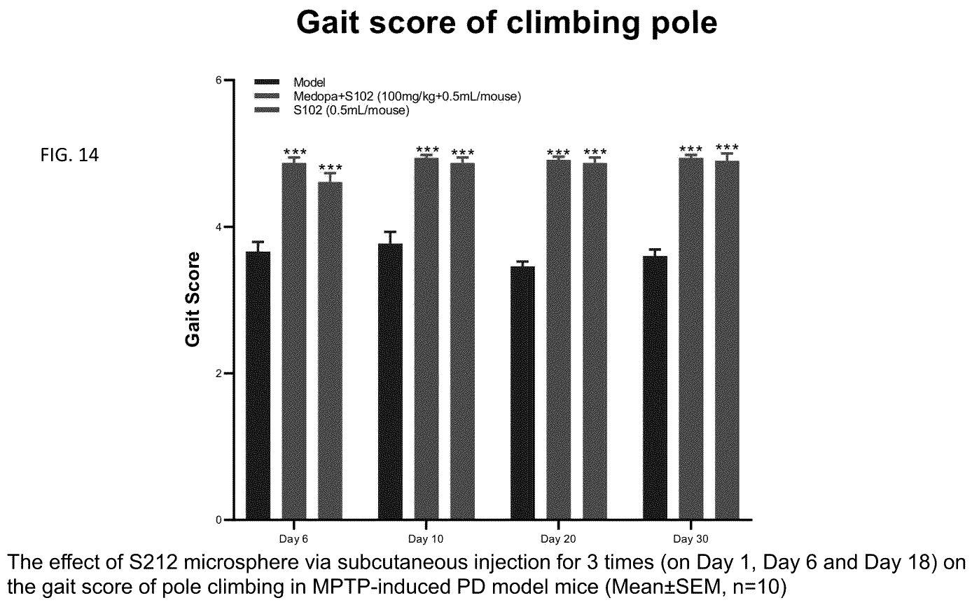

There were two main index of the pole climbing test: \textcircled{1} the time required to climb from the top to the bottom of the pole (the time of pole climbing), and \textcircled{2} the gait score of pole climbing test. \textcircled{1} The effect on the pole climbing time: according to the data in FIG. 13 and Tab.2-1, after three administrations (on Day 1, Day 6 and Day 18), compared with the model group, the climbing time of PD mice in the S212 microsphere+medopa (100 mg/kg+0.5 mL/mouse) group and S212 microsphere (0.5 mL/mouse) group decreased significantly, suggesting that the motor function damage of PD mice had certain improved after treatment. \textcircled{2} The effect on the gait score of climbing: according to the data in FIG. 14 and Tab.2-2, after three administrations (on Day 1, Day 6 and Day 18), S212 microsphere+medopa (100 mg/kg+0.5 mL/mouse) group and S212 microsphere (0.5 mL/mouse) group showed a significant increase in gait score of PD mice compared with the model group.

In summary, in the pole climbing test, S212 microsphere+medopa (100 mg/kg+0.5 mL/mouse) and S212 microsphere (0.5 mL/mouse) for 3 times (on Day 1, Day 6 and Day 18) of administration can improve the limb movement injury induced by MPTP in PD model mice, showed as shortening the time of pole climbing and increasing the gait score of the mice. See FIG. 13-14.

TABLE 2-1
The effect of S212 microsphere via subcutaneous injection for 3 times (on Day 1, Day 6 and
Day 18) on the time of pole climbing in MPTP-induced PD model mice (Mean ± SD, n = 10)

| | Time of climbing pole (s) | | | |
|---|---|---|---|---|
| Group | Day 6 | Day 10 | Day 20 | Day 30 |
| G1: Model | 10.45 ± 1.49 | 12.24 ± 1.96 | 12.03 ± 2.74 | 8.76 ± 1.52 |
| G2: Medopa + S102 | 5.12 ± 1.71*** | 5.03 ± 1.69*** | 6.12 ± 1.05*** | 4.41 ± 1.6*** |
| (100 mg/kg + 0.5 mL/mouse) | | | | |
| G3: S102 (0.5 mL/mouse) | 5.54 ± 0.46*** | 5.19 ± 1.31*** | 5.86 ± 1.22*** | 4.52 ± 0.9*** |

***P < 0.001 vs. Model

TABLE 2-2
The effect of S212 microsphere via subcutaneous injection for
for 3 times (on Day 1, Day 6 and Day 18) on the time of pole
climbing in MPTP-induced PD model mice (Mean ± SD, n = 10)

| | Gait score of climbing pole | | | |
|---|---|---|---|---|
| Group | Day 6 | Day 10 | Day 20 | Day3 0 |
| G1: Model | 3.67 ± 0.42 | 3.77 ± 0.5 | 3.47 ± 0.17 | 3.6 ± 0.26 |
| G2: Medopa + S102 | 4.87 ± 0.23*** | 4.93 ± 0.14*** | 4.9 ± 0.16*** | 4.93 ± 0.14*** |
| (100 mg/kg + 0.5 mL/mouse) | | | | |
| G3: S102 (0.5 mL/mouse) | 4.6 ± 0.38*** | 4.87 ± 0.23*** | 4.87 ± 0.23*** | 4.9 ± 0.32*** |

***P < 0.001 vs. Model

### The Effect of S212 Microsphere Via Subcutaneous Injection for 3 Times (on Day 1, Day 6 and Day 18) on the Grip Strength in MPTP-Induced PD Model Mice

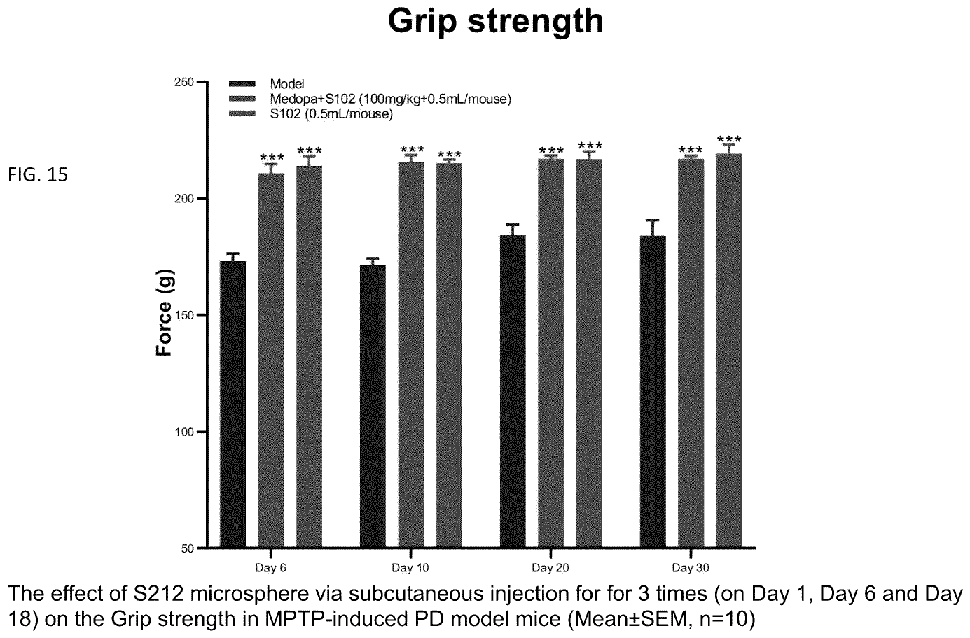

According to the data in FIG. 15 and Tab.3, compared with the model group, both the S212 microsphere+medopa (100 mg/kg+0.5 mL/mouse) and S212 microsphere (0.5 mL/mouse) significantly improved the grip strength of PD mice (P<0.001 vs Model group).

TABLE 3
The effect of S212 microsphere via subcutaneous injection for for 3 times (on Day 1, Day
6 and Day 18) on the Grip strength in MPTP-induced PD model mice (Mean ± SD, n = 10)

| | Grip strength | | | |
|---|---|---|---|---|
| Group | Day 6 | Day 10 | Day 20 | Day 30 |
| G1: Model | 173.15 ± 10.16 | 171.35 ± 9.33 | 184.25 ± 14.52 | 183.93 ± 21.39 |
| G2: Medopa + S102 | 210.77 ± 12.22*** | 215.47 ± 9.77*** | 216.93 ± 4.43*** | 216.92 ± 4.18*** |
| (100 mg/kg + 0.5 mL/mouse) | | | | |
| G3: S102 (0.5 mL/mouse) | 213.92 ± 13.43*** | 215.1 ± 5.14*** | 216.77 ± 10.62*** | 219.13 ± 13.08*** |

***P < 0.001 vs. Model

### The Effect of S212 Microsphere Via Subcutaneous Injection for 3 Times (on Day 1, Day 6 and Day18) on the Body Weight in MPTP-Induced PD Model Mice

According to the data in Tab.4, during the administration period, the body weight of PD mice rised slowly. Compared with the model group, the S212 microsphere+medopa (100 mg/kg+0.5 mL/mouse) and S212 microsphere (0.5 mL/mouse) had no significant effect on the body weight of PD mice.

TABLE 4
The effect of S212 microsphere via subcutaneous injection for 3 times (on Day 1, Day
6 and Day 18) on the body weight in MPTP-induced PD model mice (Mean ± SD, n = 10)

| | Body weight (g) | | | | |
|---|---|---|---|---|---|
| Group | Before dose | Day 4 | Day 7 | Day 11 | Day 14 |
| G1: Model | 21.86 ± 0.62 | 22.51 ± 0.61 | 22.27 ± 0.92 | 22.81 ± 0.97 | 23.26 ± 1.08 |
| G2: Medopa + S102 | 21.78 ± 0.7 | 22.03 ± 0.63 | 22.25 ± 0.85 | 22.76 ± 0.75 | 23.09 ± 0.68 |
| (100 mg/kg + 0.5 mL/mouse) | | | | | |
| G3: S102 (0.5 mL/mouse) | 21.75 ± 0.98 | 22.3 ± 0.9 | 22.68 ± 0.86 | 23.28 ± 0.87 | 23.79 ± 0.89 |

Body weight (g)

| Group | Day 18 | Day 21 | Day 25 | Day 28 |
|---|---|---|---|---|
| G1: Model | 24.18 ± 1.14 | 24.34 ± 1.21 | 25.36 ± 1.24 | 25.96 ± 1.23 |
| G2: Medopa + S102 | 24.63 ± 0.83 | 24.59 ± 0.8 | 25.9 ± 1.03 | 26.63 ± 1.24 |
| (100 mg/kg + 0.5 mL/mouse) | | | | |
| G3: S102 (0.5 mL/mouse) | 24.75 ± 1.04 | 24.76 ± 0.92 | 25.98 ± 1.05 | 26.44 ± 1.42 |

### Conclusion

In this study, S212 microsphere+medopa (100 mg/kg+0.5 mL/mouse) and S212 microsphere (0.5 mL/mouse) via subcutaneous injection for 3 times (on Day 1, Day 6, Day18) can improve the limb movement injury of PD model mice induced by MPTP.

### Example 4—Evaluation of Pharmacokinetic Profiles of L-Carnosine after Single Intramuscular Injection (IM) of S212 Microspheres in SD Rat

The purpose of this study will be to determine the pharmacokinetics profile of L-Carnosine by LC-MS/MS method in SD rat following a single intramuscular injection (IM). The groups of animals were as follows:

### Group 1

Test Article: NA; Dosage Level: NA
Vehicle: blank formulation (1 mg/mL polysorbate
20 in saline); Dosage Volume: 0.2 ml/animal
Route: intramuscular injection Theoretical Conc.: NA

| | | Supposed | Practical | | |
|---|---|---|---|---|---|
| Ingredient | ID Number | Quantity | Quantity | Preparation | |
| 1 mg/mL | 240914-02 | 2 ml | 2 ml | correct | NA |
| polysorbate 20 | | | | calculation | |
| in saline | | | | | |

The PH was measured using PH paper as/pH: ~7
Amount of Preparation: 2 ml
The physical appearance was recorded as: colorless clear solution

### Group 2

Test Article: S212-50:50 Sigma polymer; Dosage Level: 24 mg/animal
Vehicle: 1 mg/mL polysorbate 20 in saline; Dosage Volume: 0.2 ml/animal
Route: intramuscular injection; Theoretical Conc.: 12 mg/0.1 ml

| | | Supposed | Practical | | | | | | |
|---|---|---|---|---|---|---|---|---|---|
| Ingredient | ID Number | Quantity | Quantity | Preparation | | | | | |
| S212-50:50 | PK243583 | 600 mg | 450.8 mg | correct calculation | | 12/0.1*2/0.4 = 600 | | | |
| Sigma | | | | | | | | | |
| polymer | | | | | | | | | |
| | | | | Vortex | 1 min | Sonicate | NA | Clear: | N |
| 1 mg/mL | 240914-02 | 2 ml | 1.503 ml | other | stir for ~10 days | | | | |
| polysorbate 20 | | | | | | | | | |
| in saline | | | | | | | | | |

The PH was measured using PH paper as/pH: ~7
Amount of Preparation: 1.503 ml
The physical appearance was recorded as: brown suspension

### Group 3

Test Article: S212-65:35 Sigma polymer; Dosage Level: 12 mg/animal
Vehicle: 1 mg/mL polysorbate 20 in saline; Dosage Volume: 0.2 ml/animal
Route: intramuscular injection; Theoretical Conc.: 6 mg/0.1 ml

| | | Supposed | Practical | | | | | | |
|---|---|---|---|---|---|---|---|---|---|
| Ingredient | ID Number | Quantity | Quantity | Preparation | | | | | |
| S212-65:35 | PK243585 | 300 mg | 471.0 mg | correct calculation | | 6/0.1*2/0.4 = 300 | | | |
| Sigma polymer | | | | | | | | | |
| | | | | Vortex | 5 min | Sonicate | NA | Clear: | N |
| 1 mg/mL | 240914-02 | 2 ml | 3.14 ml | other | stir for ~10 days | | | | |
| polysorbate 20 | | | | | | | | | |
| in saline | | | | | | | | | |

The PH was measured using PH paper as/pH: ~7
Amount of Preparation: 3.14 ml
The physical appearance was recorded as: brown suspension

The analytical method used was as follows:

| Instrument | LC-MS/MS-28 (TQ6500+) | |
|---|---|---|
| Matrix | SD Rat Plasma | |
| Analyte(s) | L-Carnosine | |
| Internal | Wafarin (IS) | |
| standard(s) | | |
| MS conditions | APCI: Positive | |
| | MRM detection | |
| | L-Carnosine | Q1/Q3 Masses: |
| | | 227.00/110.10 Da |
| | IS: | Q1/Q3 Masses: |
| | | 309.10/163.10 Da |
| HPLC | Mobile phase: | |
| conditions | Mobile phase A: 2 mM Ammonium | |
| | acetate in 0.1% FA in water | |
| | Mobile phase B: 2 mM Ammonium formate and 0.1% FA in | |
| | acetonitrile | |
| | weak wash: 50% MeOH/Water(v:v = 1:1) | |
| | strong wash: IPA:ACN:MeOH:0.1% FA in water(v:v:v:v, 1:1:1:1) | |
| | Time (min) | Moblie phase B (%) |
| | 0.01 | 90 |
| | 0.60 | 40 |
| | 1.20 | 40 |
| | 1.21 | 90 |
| | 1.50 | 90 |
| | Column: ACQUITY UPLC BEH Amide | |
| | 1.7 um 2.1*50 mm | |
| | Oven: 40° C. | |
| | Flow rate: 0.60 mL/min | |
| | Retention time: | |
| | L-Carnosine: | 1.03 min |
| | IS: | 0.26 min |

| | |
|---|---|
| Sample | An aliquot of 20 μL plasma sample was protein precipitated with 400 μL MeOH in |
| preparation | which contains 100 ng/mL IS. The mixture was vortexed for 1 min. Then for |
| | samples treated with tube were centrifuged at 14000 rpm for 7 min, but for samples |
| | treated with 96 well plates were centrifuged at 4000 rpm for 10 min. Transfer 380 |
| | μL supernatant to 96 well plates. An aliquot of 5 μL supernatant was injected for |
| | LC-MS/MS analysis. |
| Calibration | 20-20000 ng/mL for L-Carnosine in SD Rat Plasma samples |
| curve | |

| | | |
|---|---|---|
| Instrument | LC-MS/MS-28 (TQ6500+) | |
| Matrix | SD Rat Plasma | |
| Analyte(s) | L-Carnosine | |
| Internal | Wafarin (IS) | |
| standard(s) | | |
| MS | APCI: Positive | |
| conditions | | |
| | MRM detection | |
| | L-Carnosine | Q1/Q3 Masses: |
| | | 227.00/110.10 Da |
| | IS: | Q1/Q3 Masses: |
| | | 309.10/163.10 Da |
| HPLC | Mobile phase: | |
| conditions | | |
| | Mobile phase A: 2 mM Ammonium | |
| | acetate in 0.1% FA in water | |

Mobile phase B: 2 mM Ammonium formate and 0.1% FA in
acetonitrile

weak wash: 50%
MeOH/Water(v:v = 1:1)

strong wash: IPA:ACN:MeOH:0.1% FA in water(v:v:v:v, 1:1:1:1)

| Time (min) | Mobile phase B (%) |
|---|---|
| 0.01 | 90 |
| 0.60 | 40 |
| 1.20 | 40 |
| 1.21 | 90 |
| 1.50 | 90 |
| Column: ACQUITY UPLC BEH | |
| Amide 1.7 um 2.1*50 mm | |
| Oven: 40° C. | |
| Flow rate: 0.60 mL/min | |
| Retention time: | |
| L-Carnosine: | 1.03 min |
| IS: | 0.29 min |

| | |
|---|---|
| Sample | An aliquot of 20 μL plasma sample was protein precipitated with 400 μL MeOH in |
| preparation | which contains 100 ng/mL IS. The mixture was vortexed for 1 min and centrifuged |
| | at 18000 g for 7 min. Then for samples treated with tube were centrifuged at |
| | 14000 rpm for 7 min, but for samples treated with 96 well plates were centrifuged at |
| | 4000 rpm for 10 min. Transfer 380 μL supernatant to 96 well plates. An aliquot of 5 |
| | μL supernatant was injected for LC-MS/MS analysis. |

| | |
|---|---|
| Calibration | 10-10000 ng/mL for L-Carnosine in SD Rat Plasma samples |
| curve | |

### Study Design

| Description of Test Articles | | | |
|---|---|---|---|
| Compound_ID | Correction factor | Note. | PK NO. |
| S212-50:50 | 0.4 | S212 microspheres, | PK243583 |
| Sigma polymer | | 4/6: drug/polymer | |
| | | ratio | |
| S212-65:35 | 0.4 | S212 microspheres, | PK243585 |
| Sigma polymer | | 4/6: drug/polymer | |
| | | ratio | |

| Description of Standard Articles | | | | | |
|---|---|---|---|---|---|
| Compound_ID | Batch NO. | Purity/% | Molecular_Weight | Formula_Weight | PK NO. |
| L-Carnosine | C9625-10MG | ~99% | 226.23 | NA | PK242898 |

Note:
the test article is prepared as free form concentration. The purity does not need to adjustment.

Test Article Preparation

1 mg/mL polysorbate 20 in saline.
Blank formulation preparation:
polysorbate 20:saline (w:v) =
1 mg:1 mL.
Dose preparation, for
example:
Group 2: Weigh 30 mg S212-50:50 Sigma polymer, and
add 0.1 mL blank formulation (polysorbate 20:saline
(w:v) = 1 mg:1 mL).
Group 3: Weigh 30 mg S212-65:35 Sigma polymer, and
add 0.2 mL blank formulation (polysorbate 20:saline
(w:v) = 1 mg:1 mL).

Test System

Species and Strain: SD rat, SPF.
Source: Medicilon Colony: 999M-017.
Gender: Male
Number of Animals, transferred, 20 Male rats, used, 15 Male rats.
Selection for Study: No formal randomization will be required.

Study Design

| | | | | Dose | Dose | | | |
|---|---|---|---|---|---|---|---|---|
| | | | | Conc. | Volume | Route | | |
| | | No. | Dose Level | (mg/ | (mL/ | of | | |
| Group | TA | Male** | (mg/animal) | 0.1 mL) | animal) | Dosing | Collection | Analysis |
| 1 | blank | 3 | — | — | 0.20 | IM | Plasma | L- |
| | formulation (1 | | | | | | | Carnosine |
| | mg/mL | | | | | | | |
| | polysorbate 20 | | | | | | | |
| | in saline) | | | | | | | |
| 2 | S212-50:50 | 3 | 24 | 12 | 0.20 | IM | | |
| | Sigma polymer | | | | | | | |
| 3 | S212-65:35 | 3 | 12 | 6 | 0.20 | IM | | |
| | Sigma polymer | | | | | | | |

**n = 3/time/group

Group 1˜Group 3: The test articles were be administered via intramuscular injection (IM), 0.1 mL/site*2/animal.

Collection Intervals
post-dose at Day 1-24 h, 72 h, 168 h, 336 h, 504 h, 720 h, 1080 h, 2160 h.
The blood collection sequence is shown in the following table:

| | | Animal | | | | | | | | |
|---|---|---|---|---|---|---|---|---|---|---|
| | | No./time | | | | | | | | |
| | Animal | points | 24 | 72 | 168 | 336 | 504 | 720 | 1080 | 2160 |
| Group* | ID | Male | h | h | h | h | h | h | h | h |
| n | n01-n03 | 3 | X | X | X | X | X | X | X | X |

*n = 1, 2, and 3.

### Blood Sample Collection and Procedure

The blood was taken via jugular vein or other suitable vein, 0.15 mL/time point. Sample was placed in tubes containing K2-EDTA and stored on ice until centrifuged. The blood samples was centrifuged at 6800 g for 6 minutes at 2-8° C. within 1 h after collected and stored frozen at approximately −80° C.

### Sample Analysis and Data Processing

The analytical result was confirmed using quality control samples for intra-assay variation. The accuracy of >66.7% of the quality control samples should be between 80-120% of the known value(s). Standard set of parameters including Area Under the Curve (AUC(0-t) and AUC(0-∞)), elimination half-live (T½), maximum plasma concentration (Cmax), time to reach maximum plasma concentration (Tmax) will be calculated using noncompartmental analysis modules in FDA certified pharmacokinetic program Phoenix WinNonlin 7.0 (Pharsight, USA) by the Study Director.

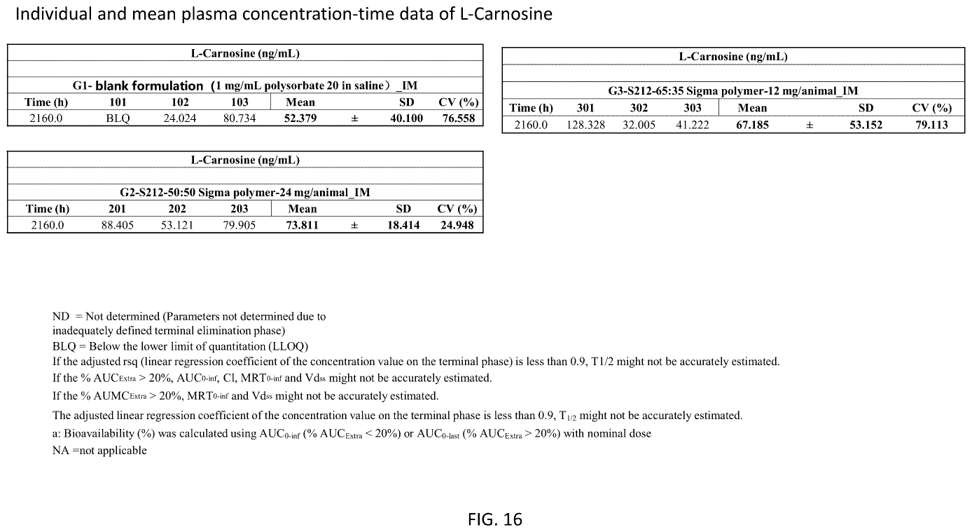

As shown in FIG. 16. Significant increases in measured L-carnosine was detected at 90 days post microsphere injection (G2, G3) compared to control (G1).

### VARIOUS EMBODIMENTS

Embodiment 1. A microsphere formulation effective for treatment of a neurological disease or condition comprising: a plurality of microspheres comprising carnosine; a biodegradable polymer; and cyclodextrin; wherein the carnosine is entrapped by the biodegradable polymer and cyclodextrin; and wherein the microspheres have an average size of less than 100 μm.

Embodiment 3. Any embodiment disclosed herein wherein the carnosine is one or more of L-carnosine, D-carnosine, acetyl-carnosine, and combinations thereof.

Embodiment 4. Any embodiment disclosed herein wherein the biodegradable polymer comprises one or more of poly(D,L-lactide), a poly(D,L-lactide-co-glycolide), a poly(ortho ester), a poly(phosphazine), a poly(phosphate ester), a polycaprolactone, a polyethylene glycol, sodium-gamma-polyglutamate, hyaluornic acid, and combinations thereof.

Embodiment 5. Any embodiment disclosed herein wherein the biodegradable polymer is a poly(D,L-lactide-co-glycolide).

Embodiment 6. Any embodiment disclosed herein wherein the biodegradable polymer is a poly(D,L-lactide-co-glycolide) having a lactide to glycolide ratio of about 75:25.

Embodiment 7. Any embodiment disclosed herein wherein the biodegradable polymer is a poly(D,L-lactide-co-glycolide) having a lactide to glycolide ratio of about 50:50.

Embodiment 8. Any embodiment disclosed herein wherein the microsphere further comprises one or more of polyvinyl alcohol, polyanhydrides, polyamines, polyesteramides, polydioxanones, polyacetals, polyketals, polycarbonates, polyphosphesters, polyethers, polyesters, polybutylene, terephthalate, polyorthocarbonates, polyphosphazenes, succinates, poly(malic acid), poly(amino acids), polyvinypyrrolidone, polysaccharides, oligosaccharides, alginate copolymers, terpolymers and combinations thereof.

Embodiment 9. Any embodiment disclosed herein wherein the microspheres have an average size of 10 μm to 50 μm.

Embodiment 10. Any embodiment disclosed herein wherein the microspheres have an average size of 10 μm to 50 μm wherein 90% of the microspheres have a size between 1 μm and 100 μm.

Embodiment 11. Any embodiment disclosed herein wherein the cyclodextrin is an alpha cyclodextrin, a beta cyclodextrin, a gamma cyclodextrin, a 2-hydroxypropyl-β-cyclodextrin, a β-cyclodextrin sulfobutylether, hydroxyethyl-β-cyclodextrin, methyl-β-cyclodextrin, dimethyl-β-cyclodextrin, carboxymethyl-β-cyclodextrin, carboxymethyl ethyl-β-cyclodextrin, diethyl-β-cyclodextrin, tri-O-alkyl-1-β-cyclodextrin, glycosyl-β-cyclodextrin, maltosyl-β-cyclodextrin or any derivative thereof, and any combination thereof.

Embodiment 12. Any embodiment disclosed herein wherein the cyclodextrin is a gamma cyclodextrin.

Embodiment 13. Any embodiment disclosed herein wherein the microsphere comprises 1%-20% cyclodextrin by weight.

Embodiment 14. Any embodiment disclosed herein wherein the microsphere comprises 1%-80% carnosine by weight.

Embodiment 15. Any embodiment disclosed herein wherein the microsphere comprises 1%-80% biodegradable polymer by weight.

Embodiment 16. A microsphere effective for treatment of a neurological disease or condition produced by the process of: providing an aqueous phase comprising carnosine in an aqueous solution; providing an organic phase comprising a biodegradable polymer and cyclodextrin in an organic solution; combining the organic phase with the aqueous phase to produce a primary emulsion; providing an external aqueous phase; combining the primary emulsion with the external aqueous phase to produce a double emulsion; and evaporating the solvents to produce the microsphere formulation.

Embodiment 17. Any embodiment disclosed herein wherein the aqueous phase further comprises one or more of polyvinyl alcohol, polyanhydrides, polyamines, polyesteramides, polydioxanones, polyacetals, polyketals, polycarbonates, polyphosphesters, polyethers, polyesters, polybutylene, terephthalate, polyorthocarbonates, polyphosphazenes, succinates, poly(malic acid), poly(amino acids), polyvinypyrrolidone, polysaccharides, oligosaccharides, alginate copolymers, terpolymers and combinations thereof.

Embodiment 18. Any embodiment disclosed herein wherein the organic phase biodegradable polymer comprises one or more of poly(D,L-lactide), a poly(D,L-lactide-co-glycolide), a poly(ortho ester), a poly(phosphazine), a poly(phosphate ester), a polycaprolactone, a polyethylene glycol, sodium-gamma-polyglutamate, hyaluornic acid, and combinations thereof.

Embodiment 19. Any embodiment disclosed herein wherein the organic phase biodegradable polymer comprises a poly(D,L-lactide-co-glycolide).

Embodiment 20. Any embodiment disclosed herein wherein the organic phase biodegradable polymer comprises a poly(D,L-lactide-co-glycolide) having a lactide to glycolide ratio of about 75:25.

Embodiment 21. Any embodiment disclosed herein wherein the organic phase biodegradable polymer comprises a poly(D,L-lactide-co-glycolide) having a lactide to glycolide ratio of about 50:50.

Embodiment 22. Any embodiment disclosed herein wherein the cyclodextrin is an alpha cyclodextrin, a beta cyclodextrin, a gamma cyclodextrin, a 2-hydroxypropyl-β-cyclodextrin, a β-cyclodextrin sulfobutylether, hydroxyethyl-β-cyclodextrin, methyl-β-cyclodextrin, dimethyl-β-cyclodextrin, carboxymethyl-β-cyclodextrin, carboxymethyl ethyl-β-cyclodextrin, diethyl-β-cyclodextrin, tri-O-alkyl-1-β-cyclodextrin, glycosyl-β-cyclodextrin, maltosyl-β-cyclodextrin or any derivative thereof, and any combination thereof.

Embodiment 23. Any embodiment disclosed herein wherein the cyclodextrin is a gamma cyclodextrin.

Embodiment 24. Any embodiment disclosed herein wherein the microsphere comprises 1%-20% cyclodextrin by weight.

Embodiment 25. Any embodiment disclosed herein wherein the microsphere comprises 1%-80% carnosine by weight.

Embodiment 26. Any embodiment disclosed herein wherein the microsphere comprises 1%-80% biodegradable polymer by weight.

Embodiment 27. A method of treating or ameliorating a neurodegenerative disease or condition in a subject in need thereof comprising the step administering to the subject the formulation of any embodiment herein or the microsphere of any embodiment herein.

Embodiment 28. Any embodiment disclosed herein wherein the formulation or microsphere is administered orally, intramuscularly, intravenously, subcutaneously, intrathecally, and/or by nasal spray.

Embodiment 29. Any embodiment disclosed herein wherein the formulation or microsphere is administered no more than once per week.

Embodiment 30. Any embodiment disclosed herein wherein the formulation or microsphere is administered no more than once per month.

Embodiment 31. Any embodiment disclosed herein wherein the formulation or microsphere is administered no more than once every six months.

Embodiment 32. A method of making a microsphere effective for treatment of a neurological disease or comprising the steps of: providing an aqueous phase comprising carnosine in an aqueous solution; providing an organic phase comprising a biodegradable polymer and cyclodextrin in an organic solution; combining the organic phase with the aqueous phase to produce a primary emulsion; providing an external aqueous phase; combining the primary emulsion with the external aqueous phase to produce a double emulsion; and evaporating the solvents to produce the microsphere formulation.

Embodiment 33. Any embodiment disclosed herein wherein the aqueous phase further comprises one or more of polyvinyl alcohol, polyanhydrides, polyamines, polyesteramides, polydioxanones, polyacetals, polyketals, polycarbonates, polyphosphesters, polyethers, polyesters, polybutylene, terephthalate, polyorthocarbonates, polyphosphazenes, succinates, poly(malic acid), poly(amino acids), polyvinypyrrolidone, polysaccharides, oligosaccharides, alginate copolymers, terpolymers and combinations thereof.

Embodiment 34. Any embodiment disclosed herein wherein the organic phase biodegradable polymer comprises one or more of poly(D,L-lactide), a poly(D,L-lactide-co-glycolide), a poly(ortho ester), a poly(phosphazine), a poly(phosphate ester), a polycaprolactone, a polyethylene glycol, sodium-gamma-polyglutamate, hyaluornic acid, and combinations thereof.

Embodiment 35. Any embodiment disclosed herein wherein the organic phase biodegradable polymer comprises a poly(D,L-lactide-co-glycolide).

Embodiment 36. Any embodiment disclosed herein wherein the organic phase biodegradable polymer comprises a poly(D,L-lactide-co-glycolide) having a lactide to glycolide ratio of about 75:25.

Embodiment 37. Any embodiment disclosed herein wherein the organic phase biodegradable polymer comprises a poly(D,L-lactide-co-glycolide) having a lactide to glycolide ratio of about 50:50.

Embodiment 38. Any embodiment disclosed herein wherein the cyclodextrin is an alpha cyclodextrin, a beta cyclodextrin, a gamma cyclodextrin, a 2-hydroxypropyl-β-cyclodextrin, a β-cyclodextrin sulfobutylether, hydroxyethyl-β-cyclodextrin, methyl-β-cyclodextrin, dimethyl-β-cyclodextrin, carboxymethyl-β-cyclodextrin, carboxymethyl ethyl-β-cyclodextrin, diethyl-β-cyclodextrin, tri-O-alkyl-1-β-cyclodextrin, glycosyl-β-cyclodextrin, maltosyl-β-cyclodextrin or any derivative thereof, and any combination thereof.

Embodiment 39. Any embodiment disclosed herein wherein the cyclodextrin is a gamma cyclodextrin.

Embodiment 40. Any embodiment disclosed herein wherein the microsphere comprises 1%-20% cyclodextrin by weight.

Embodiment 41. Any embodiment disclosed herein wherein the microsphere comprises 1%-80% carnosine by weight.

Embodiment 42. Any embodiment disclosed herein wherein the microsphere comprises 1%-80% biodegradable polymer by weight.

Embodiment 43. Any embodiment disclosed herein wherein the microspheres are effective to encapsulate at least 20% of the peptide.

Embodiment 44. Any embodiment disclosed herein wherein the microspheres are effective to encapsulate at least 40% of the peptide.

Embodiment 45. Any embodiment disclosed herein wherein the microspheres are effective to encapsulate at least 60% of the peptide.

Embodiment 46. Any embodiment disclosed herein wherein the microspheres are effective to encapsulate at least 80% of the peptide.

Embodiment 47. Any embodiment disclosed herein wherein the microspheres are effective to encapsulate at least 20% of the peptide.

Embodiment 48. Any embodiment disclosed herein wherein the microspheres are effective to encapsulate at least 40% of the peptide.

Embodiment 49. Any embodiment disclosed herein wherein the microspheres are effective to encapsulate at least 60% of the peptide.

Embodiment 50. Any embodiment disclosed herein wherein the microspheres are effective to encapsulate at least 80% of the peptide.

Embodiment 51. Any embodiment disclosed herein wherein the microspheres are effective to encapsulate at least 20% of the peptide.

Embodiment 52. Any embodiment disclosed herein wherein the microspheres are effective to encapsulate at least 40% of the peptide.

Embodiment 53. Any embodiment disclosed herein wherein the microspheres are effective to encapsulate at least 60% of the peptide.

Embodiment 54. Any embodiment disclosed herein wherein the microspheres are effective to encapsulate at least 80% of the peptide.

Embodiment 55. Any embodiment disclosed herein further comprising a lyoprotectant.

Embodiment 56. Any embodiment disclosed herein wherein the microsphere further comprises a lyoprotectant.

Embodiment 57. Any embodiment disclosed herein wherein the microsphere further comprises a lyoprotectant.

Embodiment 58. A method of treating or ameliorating a neurodegenerative disease or condition in a subject in need thereof comprising the step of administering to the subject a microsphere formulation comprising: a plurality of microspheres comprising carnosine; a biodegradable polymer; and cyclodextrin; wherein the carnosine is entrapped by the biodegradable polymer and cyclodextrin; and wherein the microspheres have an average size of less than 100 μm.

Embodiment 59. Any embodiment disclosed herein wherein the carnosine is one or more of L-carnosine, D-carnosine, acetyl-carnosine, and combinations thereof.

Embodiment 60. Any embodiment disclosed herein wherein the biodegradable polymer comprises one or more of poly(D,L-lactide), a poly(D,L-lactide-co-glycolide), a poly(ortho ester), a poly(phosphazine), a poly(phosphate ester), a polycaprolactone, a polyethylene glycol, sodium-gamma-polyglutamate, hyaluornic acid, and combinations thereof.

Embodiment 61. Any embodiment disclosed herein wherein the biodegradable polymer is a poly(D,L-lactide-co-glycolide).

Embodiment 62. Any embodiment disclosed herein wherein the biodegradable polymer is a poly(D,L-lactide-co-glycolide) having a lactide to glycolide ratio of about 75:25.

Embodiment 63. Any embodiment disclosed herein wherein the biodegradable polymer is a poly(D,L-lactide-co-glycolide) having a lactide to glycolide ratio of about 50:50.

Embodiment 64. Any embodiment disclosed herein wherein the microsphere further comprises one or more of polyvinyl alcohol, polyanhydrides, polyamines, polyesteramides, polydioxanones, polyacetals, polyketals, polycarbonates, polyphosphesters, polyethers, polyesters, polybutylene, terephthalate, polyorthocarbonates, polyphosphazenes, succinates, poly(malic acid), poly(amino acids), polyvinypyrrolidone, polysaccharides, oligosaccharides, alginate copolymers, terpolymers and combinations thereof.

Embodiment 65. Any embodiment disclosed herein wherein the microspheres have an average size of 10 μm to 50 μm.

Embodiment 66. Any embodiment disclosed herein wherein the microspheres have an average size of 10 μm to 50 μm wherein 90% of the microspheres have a size between 1 μm and 100 μm.

Embodiment 67. Any embodiment disclosed herein wherein the cyclodextrin is an alpha cyclodextrin, a beta cyclodextrin, a gamma cyclodextrin, a 2-hydroxypropyl-β-cyclodextrin, a β-cyclodextrin sulfobutylether, hydroxyethyl-β-cyclodextrin, methyl-β-cyclodextrin, dimethyl-β-cyclodextrin, carboxymethyl-β-cyclodextrin, carboxymethyl ethyl-β-cyclodextrin, diethyl-β-cyclodextrin, tri-O-alkyl-1-β-cyclodextrin, glycosyl-β-cyclodextrin, maltosyl-β-cyclodextrin or any derivative thereof, and any combination thereof.

Embodiment 68. Any embodiment disclosed herein wherein the cyclodextrin is a gamma cyclodextrin.

Embodiment 69. Any embodiment disclosed herein wherein the microsphere comprises 1%-20% cyclodextrin by weight.

Embodiment 70. Any embodiment disclosed herein wherein the microsphere comprises 1%-80% carnosine by weight.

Embodiment 71. Any embodiment disclosed herein wherein the microsphere comprises 1%-80% biodegradable polymer by weight.

Embodiment 72. Any embodiment disclosed herein wherein the formulation is administered orally, intramuscularly, intravenously, subcutaneously, intrathecally, and/or by nasal spray.

Embodiment 73. Any embodiment disclosed herein wherein the formulation is administered no more than once per week.

Embodiment 74. Any embodiment disclosed herein wherein the formulation is administered no more than once per month.

Embodiment 75. Any embodiment disclosed herein wherein the formulation is administered no more than once every six months.

Embodiment 76. Any embodiment disclosed herein wherein the neurodegenerative disease or condition is Alzheimer's disease.

Embodiment 77. Any embodiment disclosed herein wherein the neurodegenerative disease or condition is Parkinson's disease.

Embodiment 78. Any embodiment disclosed herein wherein the microspheres are effective to encapsulate at least 20% of the carnosine.

Embodiment 79. Any embodiment disclosed herein wherein the microspheres are effective to encapsulate at least 40% of the carnosine.

Embodiment 80. Any embodiment disclosed herein wherein the microspheres are effective to encapsulate at least 60% of the carnosine.

Embodiment 81. Any embodiment disclosed herein wherein the microspheres are effective to encapsulate at least 80% of the carnosine.

Embodiment 82. A microsphere effective for treatment of a neurodegenerative disease or condition produced by the steps of: providing an aqueous phase comprising carnosine in an aqueous solution; providing an organic phase comprising a biodegradable polymer and cyclodextrin in an organic solution; combining the organic phase with the aqueous phase to produce a primary emulsion; providing an external aqueous phase; combining the primary emulsion with the external aqueous phase to produce a double emulsion; and evaporating the solvents to produce the microsphere formulation.

Embodiment 83. Any embodiment disclosed herein wherein the aqueous phase further comprises one or more of polyvinyl alcohol, polyanhydrides, polyamines, polyesteramides, polydioxanones, polyacetals, polyketals, polycarbonates, polyphosphesters, polyethers, polyesters, polybutylene, terephthalate, polyorthocarbonates, polyphosphazenes, succinates, poly(malic acid), poly(amino acids), polyvinypyrrolidone, polysaccharides, oligosaccharides, alginate copolymers, terpolymers and combinations thereof.

Embodiment 84. Any embodiment disclosed herein wherein the organic phase biodegradable polymer comprises a poly(D,L-lactide-co-glycolide) having a lactide to glycolide ratio of about 75:25 to about 50:50.

Embodiment 85. Any embodiment disclosed herein wherein the cyclodextrin is an alpha cyclodextrin, a beta cyclodextrin, a gamma cyclodextrin, a 2-hydroxypropyl-β-cyclodextrin, a β-cyclodextrin sulfobutylether, hydroxyethyl-β-cyclodextrin, methyl-β-cyclodextrin, dimethyl-β-cyclodextrin, carboxymethyl-β-cyclodextrin, carboxymethyl ethyl-β-cyclodextrin, diethyl-β-cyclodextrin, tri-O-alkyl-1-β-cyclodextrin, glycosyl-β-cyclodextrin, maltosyl-β-cyclodextrin or any derivative thereof, and any combination thereof.

Embodiment 86. Any embodiment disclosed herein wherein the microsphere comprises 1%-20% cyclodextrin by weight.

Embodiment 87. Any embodiment disclosed herein wherein the microsphere comprises 1%-80% carnosine by weight.

All documents cited in this application are hereby incorporated by reference as if recited in full herein. In the event of a conflict between the teachings of this application and those of the incorporated documents, the teachings of this application control.

The embodiments described in this disclosure can be combined in various ways. Any aspect or feature that is described for one embodiment can be incorporated into any other embodiment mentioned in this disclosure. While various novel features of the inventive principles have been shown, described and pointed out as applied to particular embodiments thereof, it should be understood that various omissions and substitutions and changes can be made by those skilled in the art without departing from the spirit of this disclosure. Those skilled in the art will appreciate that the inventive principles can be practiced in other than the described embodiments, which are presented for purposes of illustration and not limitation.

1. A method of treating or ameliorating a neurodegenerative disease or condition in a subject in need thereof comprising the step of administering to the subject a microsphere formulation comprising:
a plurality of microspheres comprising carnosine; a biodegradable polymer; and cyclodextrin; wherein the carnosine is entrapped by the biodegradable polymer and cyclodextrin; and
wherein the microspheres have an average size of less than 100 μm.

2. The method of claim 1, wherein the carnosine is one or more of L-carnosine, D-carnosine, acetyl-carnosine, and combinations thereof.

3. The method of claim 1, wherein the biodegradable polymer comprises one or more of poly(D,L-lactide), a poly(D,L-lactide-co-glycolide), a poly(ortho ester), a poly(phosphazine), a poly(phosphate ester), a polycaprolactone, a polyethylene glycol, sodium-gamma-polyglutamate, hyaluornic acid, and combinations thereof.

4. The method of claim 1, wherein the biodegradable polymer is a poly(D,L-lactide-co-glycolide).

5. The method of claim 1, wherein the biodegradable polymer is a poly(D,L-lactide-co-glycolide) having a lactide to glycolide ratio of about 75:25.

6. The method of claim 1, wherein the biodegradable polymer is a poly(D,L-lactide-co-glycolide) having a lactide to glycolide ratio of about 50:50.

7. The method of claim 1, wherein the microsphere further comprises one or more of polyvinyl alcohol, polyanhydrides, polyamines, polyesteramides, polydioxanones, polyacetals, polyketals, polycarbonates, polyphosphesters, polyethers, polyesters, polybutylene, terephthalate, polyorthocarbonates, polyphosphazenes, succinates, poly(malic acid), poly(amino acids), polyvinypyrrolidone, polysaccharides, oligosaccharides, alginate copolymers, terpolymers and combinations thereof.

8. The method of claim 1, wherein the microspheres have an average size of 10 μm to 50 μm.

9. The method of claim 1, wherein the microspheres have an average size of 10 μm to 50 μm wherein 90% of the microspheres have a size between 1 μm and 100 μm.

10. The method of claim 1, wherein the cyclodextrin is an alpha cyclodextrin, a beta cyclodextrin, a gamma cyclodextrin, a 2-hydroxypropyl-β-cyclodextrin, a β-cyclodextrin sulfobutylether, hydroxyethyl-β-cyclodextrin, methyl-β-cyclodextrin, dimethyl-β-cyclodextrin, carboxymethyl-β-cyclodextrin, carboxymethyl ethyl-β-cyclodextrin, diethyl-β-cyclodextrin, tri-O-alkyl-1-β-cyclodextrin, glycosyl-β-cyclodextrin, maltosyl-β-cyclodextrin or any derivative thereof, and any combination thereof.

11. The method of claim 1, wherein the cyclodextrin is a gamma cyclodextrin.

12. The method of claim 1, wherein the microsphere comprises 1%-20% cyclodextrin by weight.

13. The method of claim 1, wherein the microsphere comprises 1%-80% carnosine by weight.

14. The method of claim 1, wherein the microsphere comprises 1%-80% biodegradable polymer by weight.

15. The method of claim 1, wherein the formulation is administered orally, intramuscularly, intravenously, subcutaneously, intrathecally, and/or by nasal spray.

16. The method of claim 1, wherein the formulation is administered no more than once per week.

17. The method of claim 1, wherein the formulation is administered no more than once per month.

18. The method of claim 1, wherein the formulation is administered no more than once every six months.

19. The method of claim 1, wherein the neurodegenerative disease or condition is Alzheimer's disease.

20. The method of claim 1, wherein the neurodegenerative disease or condition is Parkinson's disease.

21. The method of claim 1, wherein the microspheres are effective to encapsulate at least 20% of the carnosine.

22. The method of claim 1, wherein the microspheres are effective to encapsulate at least 40% of the carnosine.

23. The method of claim 1, wherein the microspheres are effective to encapsulate at least 60% of the carnosine.

24. The method of claim 1, wherein the microspheres are effective to encapsulate at least 80% of the carnosine.

25. A microsphere effective for treatment of a neurodegenerative disease or condition produced by the steps of:
providing an aqueous phase comprising carnosine in an aqueous polyvinyl alcohol (PVA) solution;
providing an organic phase comprising a poly(lactic-co-glycolic) acid (PLGA) polymer and cyclodextrin in a dichloromethane (DCM) solution;
combining the organic phase with the aqueous phase to produce a primary emulsion;
providing an external aqueous PVA solution;
combining the primary emulsion with the external aqueous PVA solution to produce a double emulsion; and
evaporating the solvents to produce the microsphere formulation, wherein the microspheres have an average size of less than 100 μm.

26. (canceled)

27. The microsphere of claim 25, wherein the organic phase biodegradable polymer comprises PLGA having a lactide to glycolide ratio of about 75:25 to about 50:50.

28. The microsphere of claim 25, wherein the cyclodextrin is an alpha cyclodextrin, a beta cyclodextrin, a gamma cyclodextrin, a 2-hydroxypropyl-β-cyclodextrin, a β-cyclodextrin sulfobutylether, hydroxyethyl-β-cyclodextrin, methyl-β-cyclodextrin, dimethyl-β-cyclodextrin, carboxymethyl-β-cyclodextrin, carboxymethyl ethyl-β-cyclodextrin, diethyl-β-cyclodextrin, tri-O-alkyl-1-β-cyclodextrin, glycosyl-β-cyclodextrin, maltosyl-β-cyclodextrin, any derivative thereof, or any combination thereof.

29. The microsphere of claim 25, wherein the microsphere comprises 1%-20% cyclodextrin by weight.

30. The microsphere of claim 25, wherein the microsphere comprises 1%-80% carnosine by weight.
